(19) United States
(12) Patent Application Publication
(10) Pub. No.: US 2018/0261310 A1
(43) Pub. Date: Sep. 13, 2018

(54) APPARATUS AND METHOD OF IDENTIFYING AND MONITORING A SURGICAL RISK FACTOR AND PROVIDING A TREATMENT REGIMEN FOR A PATIENT

(71) Applicant: RMFx, Inc., Denver, CO (US)

(72) Inventors: Jerome R. Edwards, Erie, CO (US); Thomas Kurian, Austin, TX (US)

(73) Assignee: RMFx, Inc., Denver, CO (US)

(21) Appl. No.: 15/919,647

(22) Filed: Mar. 13, 2018

Related U.S. Application Data
(60) Provisional application No. 62/470,563, filed on Mar. 13, 2017.

(51) Int. Cl. G16H 20/00; G16H 40/67

(52) U.S. Cl. CPC G16H 20/00; G16H 15/00; G16H 40/67

(57) ABSTRACT

A system and method of remotely monitoring health factors for patients scheduled for surgery and providing a health management protocol is provided. More specifically, the present invention relates to systems and methods of receiving biometric data about a patient, comparing the data to data related to a RFM regimen prescribed for the patient, and providing reports on the patient's progress to one or more users prior to the implementation of the surgical procedure. The users may include, but are not limited to, medical care providers, hospital administrators, and insurance providers.

### CROSS-REFERENCE TO RELATED APPLICATIONS

This application claims priority under 35 U.S.C. § 119(e) to U.S. Provisional Patent Application Ser. No. 62/470,563 filed Mar. 13, 2017, which is incorporated herein in its entirety by reference.

### FIELD OF THE INVENTION

The present invention relates generally to systems and methods that aid physicians in preparing their patients for surgery and monitoring them post-operatively to ensure the best clinical outcomes. More specifically, the invention relates to systems and methods for monitoring patient attributes in accordance with specific regimens to prepare those patients for surgery and the best published success rates associated with those surgical procedures.

### BACKGROUND OF THE INVENTION

The use of various digital health and patient monitoring technologies has become ubiquitous across the healthcare ecosystem. Smart watches, physiologic monitoring devices, apps, and published medical literature for specific chronic disease management are all available to providers and consumers of healthcare via the internet.

Smartphones and associated peripheral biometric monitoring technologies have become an avenue in which healthcare providers can remotely monitor and manage patients. Utilization of cloud-based data storage systems with electronic medical records integrated with mobile health platforms provide a platform in which large populations with any chronic disease state can be remotely monitored and interventions pursued based on biometric data obtained over time. The use of electronic medical records allows for tracking and data analysis of large cohorts of patients with specific disease states and evaluation of outcomes with respect to intervention over time. This provides a platform for at risk patients to be identified based on pre-set, guideline based biometric variables. However, no mechanism exists that utilizes these complementary technologies, devices and information sources in a tangible way to produce better patient outcomes or healthcare cost reduction in association with surgical procedures. Currently there are a number of health-based applications that are available to patients and health care providers; however, most tools currently developed only serve to store biometric data. Accordingly, it remains up to the patient to use the stored biometric data to invest in his or her own health.

Chronic disease states or non-communicable diseases are the leading cause of mortality and morbidity globally. Suboptimal lifestyle choices largely contribute to the development and progression of chronic disease in patients. Interventions that modify traditional risk factors (including diet, exercise, weight loss, tobacco cessation, and medication compliance, among others) are effective in improving clinical outcomes. Unfortunately, patients generally poorly adhere to recommendations to modify risk factors, even when prescribed as prerequisites to surgery.

Currently there is no mechanism to adequately incentivize patients to pursue positive health care choices and lifestyle risk factor modification to improve their own clinical outcome. Importantly, there is no mechanism for adjudication of patients based on their compliance to prescribed positive health regimens prior to surgery. Digital health technology today is diffuse in nature and not tied to specific disease states and associated patient regimens. Healthcare systems currently pay providers for services rendered regardless of success and impact of the service (fee for service or fee for surgery). However, the economics of healthcare are moving towards outcome based reimbursement models with fixed reimbursement assigned to a specific disease state. Further, penalties are being assessed for services (surgeries) rendered outside of standard success and complication rates.

One example of a chronic disease state in which aggressive risk factor modification is noted to improve outcomes is atrial fibrillation. Atrial fibrillation is an electrical disorder of the atria. Patients affected with this disorder have increased risk of heart failure, hospitalization, thromboembolic events including stroke and death. Symptoms associated with atrial fibrillation, which include palpitations, fatigue, and dizziness, can be debilitating to patients and may result in an overall poor quality of life. Further, atrial fibrillation has been associated with dementia as a result of micro-emboli (clots) travelling to the brain over long periods of time.

Atrial fibrillation is the most common cardiac arrhythmia worldwide. One study stated that atrial fibrillation affected an estimated 2-6 million individuals is the U.S. in 2010. This is projected to increase to 15-20 million individuals by 2050. The costs to the U.S. healthcare system were estimated at $6 billion in 2005 and are expected to continue to grow.

One method of treating atrial fibrillation is a surgery called catheter ablation. Catheter ablation has become the standard of care as a treatment option in patients with symptomatic atrial fibrillation. Unfortunately, long term outcomes of catheter ablation in patients with atrial fibrillation are noted to decline with time. Several risk factors, including hypertension, diabetes, obesity, obstructive sleep apnea, and excessive alcohol, have been linked to development of atrial fibrillation and recurrences after treatment by catheter ablation.

Risk factor modification has been shown in published medical literature to alter cardiac structures including the atria in a positive fashion resulting in lower atrial fibrillation burden and improvement of outcomes in patients post catheter ablation. Risk factor modification includes one or more of: (a) management of sleep-disordered breathing (such as sleep apnea) through the use of a continuous positive airway pressure (CPAP) device, (b) treatment of diabetes and hypertension (HTN) through medication, diet, exercise, weight loss, tobacco cessation, and (c) alcohol limitation. Previous studies as noted by P. Sanders et al. including “Aggressive Risk Factor Reduction Study for Atrial Fibrillation and Implications for the Outcome of Ablation,” Volume 64, Issue 21, ISSN 0735-1097, December 2014 and available at http://www.onlinejacc.org/content/accj/64/21/2222.full.pdf (hereinafter the “ARREST-AF article”) and “Long-Term Effect of Goal-Directed Weight Management in an Atrial Fibrillation Cohort: A Long-Term Follow-Up Study,” Volume 65, Issue 20, ISSN 0735-109, May 2015 and available at http://www.onlinejacc.org/content/accj/65/20/2159.full.pdf (hereinafter the “LEGACY article”) evaluated aggressive risk factor modification in patients with atrial fibrillation. Each of these studies is incorporated herein by reference in its entirety. The results of these studies underscored striking improvement with 5-fold improvement of outcomes when aggressive risk factor modification was pursued in addition to treatment of atrial fibrillation by catheter ablation. These studies were performed in multidisciplinary clinics. Patients in the study were evaluated in established physical clinics, tested, and their progress along prescribed regimens and the condition of their various risk factor states was tracked. While proving to be a very successful model with aggressive risk factor modification and improvement of outcomes post catheter ablation for atrial fibrillation, implementation of this approach has not been pursued on a large scale and capitalized upon due to challenges of large scale implementation.

Accordingly, there is an unmet need for a system to monitor patients remotely during their participation in a prescribed RFM regimen. More specifically, there is an unmet need for a system that receives biometric data about a patient, compares the data to rules related to a RFM regimen prescribed for the patient, and provides a report on the patient's progress to one or more users.

### SUMMARY OF THE INVENTION

It is one aspect of the present invention to provide a risk factor management system (hereinafter “RFM system”) to remotely monitor a patient in a prescribed risk factor modification program or regimen (hereinafter “RFM regimen”). The RFM system receives biometric data associated with the patient. The data may be automatically collected or entered by the patient.

In one embodiment, the RFM system includes a sensor associated with the patient. The sensor is operable to record biometric data of the patient. In one embodiment, the RFM system includes a plurality of sensors operable to record biometric information including, but not limited to, one or more of: blood pressure, weight, heart rate, heart rhythm, blood (or breath) alcohol level, respiration, nicotine, glucose level, and temperature. The sensors may include one or more of a blood sensor, a motion sensor, a thermometer, a scale, and a calorie counter.

In one embodiment, the sensors may be incorporated in a device worn or operated by patient. For example, in one embodiment, at least one of the sensors is associated with a smart device such as a smart phone, smart watch, and a fitness monitor or activity tracker. Optionally, the sensor may be adapted to be affixed to a patient or implanted in a patient.

The biometric data recorded by the sensor may be stored in memory associated with the sensor. In one embodiment, the recorded data is transmitted to a remote storage. The remote storage may be accessible by a network connection such as the internet. In one embodiment, remote storage is a cloud based storage. In another embodiment, the recorded data is stored in a database. Optionally, the database may be local to the patient or another user.

The recorded data may be transmitted to a server by a connection to a communication network, such as the internet. In one embodiment, the recorded data is transmitted to the server at predetermined times. Additionally, or alternatively, the recorded data may be transmitted to the server substantially simultaneously upon being collected by the sensor. The sensor may communicate with the network by one or more wired or wireless connections. In one embodiment, a sensor may communicate with the network by WiFi, Bluetooth, or any other wireless communication modality.

The RFM system may also receive data entered by the patient. In one embodiment, the patient may enter data related to compliance with one or more activities prescribed as part of the RFM regimen. Accordingly, the patient may periodically report information related to one or more of the patient's diet, exercise activity, tobacco use, alcohol use, medication compliance, management of sleep-disordered breathing, weight, blood pressure, and the like. The patient may enter the information in a user interface generated by the system.

The individual and composite biometric variables stored in the database can be tracked over time. The RFM system can analyze the biometric data and provide information to one or more users, including a patient, a medical care provider, a hospital administrator, an insurance provider, and a government employee. In one embodiment, the information provided to the users includes providing feedback to the patient. The feedback may include data related to the patient's progress in the RFM regimen, including recommended actions to progress in the prescribed RFM regimen. Optionally, the feedback may include an estimated reduction in the patient's insurance deductible or co-payment requirement for a surgical procedure based on the patient's progress in the RFM regimen. In one embodiment, the system may provide information to one or more of the users related to the patient's physical activity, such as compliance with prescribed risk factor modification activities.

It is another aspect of the present invention to provide a RFM system that provides information to users regarding a patient's participation in a RFM regimen. The RFM system receives biometric information related to the patient, analyzes the biometric information, and then compares the information to rules associated with elements of the RFM regimen. The RFM system may automatically generate reports related to the patient's progress. Additionally, or alternatively, the RFM system may generate reports upon demand from a user. In this manner, the RFM system provides a web and application-based platform for delineating and adjudicating readiness for medical procedures in which outcomes are improved upon with risk factor modification. Further, the RFM system will provide an interface with relevant biometric data that is available to patients, healthcare providers, and payers in the healthcare ecosystem. Accordingly, the RFM system of the present invention encourages shared patient accountability and responsibility in regard to healthcare interventions and resources.

It is another aspect of the present invention to provide a RFM system that provides incentives to a patient participating in a RFM regimen. In one embodiment, the patient receives a financial incentive for participating in the RFM regimen. In another embodiment, as the patient progresses in the RFM regimen, the financial burden of a surgical procedure for the patient decreases. The RFM system may provide information related to the patient's progress in the RFM regimen. The patient's progress may be analyzed based on rules associated with elements of the RFM regimen. The patient's progress may be assigned a numeric value or percentage, for example from 0% to 100% completion. In one embodiment, the patient's co-payment or deductible for a surgical procedure is reduced by an amount related to the percentage of the patient's progress in the RFM regimen. Optionally, when a patient completes an RFM regiment, the patient's co-payment or deductible for the surgical procedure can be reduced by 50% or more. In another embodiment, the patient's co-payment or deductible is increased if the patient does not complete an RFM regimen. In one embodiment, the patient's co-payment or deductible is increased by the percentage of the patient's RFM regimen that is not competed. For example, if the patient is 50% complete with an RFM regiment, the patient's co-payment or deductible will be increased by 50%.

One aspect of the present invention is a system for monitoring a plurality of health parameters associated with a surgical patient to improve a surgical outcome. The system includes, but is not limited to: (1) a data monitor for the surgical patient, including at least two of: a blood pressure data measurement device; a weight data measurement device; a physical activity data monitoring device; a heart rate data monitor; a blood data measurement device; and a nicotine presence data measurement device; and (2) a management system to receive data from the patient, wherein the management system is configured to store a pre-surgery regimen prescribed for the surgical patient, and wherein the management system is further configured to provide a recommendation as to the patient's readiness for the surgical procedure.

Optionally, the data monitor is an implantable medical device. In one embodiment, the data monitor is associated with a patient system. The patient system is in communication with the management system. The patient system may be a mobile device such as a smart-phone, a laptop computer, or a tablet computer. In another embodiment, the management system is a computer server accessible remotely through a network, such as the interne. Optionally, the blood data measurement device may determine a blood alcohol level, a blood glucose level, and a blood cholesterol level.

In one embodiment, the management system is further configured to receive data from the patient post-surgery. In another embodiment, the management system is operable generate post-surgery patient status reports.

In one embodiment, the pre-surgery regimen is prescribed by a medical provider. In another embodiment, the pre-surgery regimen is generated at least partially by the management system. In one embodiment, the pre-surgery regimen includes dietary instructions.

Yet another aspect of the present invention is to provide a method of improving the success of a surgical procedure for a patient. The method comprises, but is not limited to: (1) providing a health regimen for a patient who requires surgery, the health regimen designed to improve the patient's health and reduce risk associated with surgery; (2) storing, by a management system, the health regimen provided for the patient; (3) receiving data from a data monitor related to the patient, the data monitor operable to measure at least two of: (i) blood pressure; (ii) weight; (iii) physical activity; (iv) heart rate data; (v) blood alcohol data; and (vi) nicotine presence; and (4) providing a summary as to the patient's readiness for the surgery. Optionally, the data monitor may be further operable to determine blood glucose levels and blood cholesterol levels.

In another aspect of the present invention, a non-transitory computer readable media is provided. The non-transitory computer readable media has instructions stored thereon which, when executed by a processor of a management system, cause the processor to perform a method of improving the success of a surgical procedure for a patient. The instructions include: (1) an instruction to store, by the management system, a health regimen for a patient who requires surgery, the health regimen designed to improve the patient's health and reduce risk associated with surgery; (2) an instruction to receive, by the management system, data from a data monitor related to the patient, the data monitor operable to measure at least two of: (i) blood pressure; (ii) weight; (iii) physical activity; (iv) heart rate data; (v) blood alcohol data; and (vi) nicotine presence; and (3) an instruction to provide a summary as to the patient's readiness for the surgery. In one embodiment, the data monitor is operable to determine one or more of a blood glucose level, a blood cholesterol level, and the like.

One aspect of the present invention is a system for monitoring a plurality of health parameters associated with a patient to improve a surgical outcome. The system generally includes, but is not limited to: (1) a processor; and (2) a memory coupled with and readable by the processor, the memory storing therein instructions which, when executed by the processor, cause the processor to process a medical record in a database for the patient by: (i) storing a risk factor management (RFM) regimen for the patient in the medical record, the RFM regimen identifying at least one health parameter requiring an action by the patient; (ii) receiving data related to the patient's RFM regimen from a data monitor, the data comprising at least one of: the patient's blood pressure, the patient's weight, an amount of physical activity performed by the patient, the patient's heart rate, an amount of alcohol consumed by the patient, and an amount of tobacco consumed by the patient; and (iii) determining when the patient is ready for surgery. In one embodiment, the system further comprises receiving data related to the patient post-surgery. In another embodiment, the system further comprises generating a post-surgery patient status report. In one embodiment, the RFM regimen includes at least one of: a dietary regimen, an exercise program, a medication protocol, a target blood pressure, a target blood glucose level, lipid management, arrhythmia management, a reduction in alcohol consumption, a reduction in tobacco use, a target weight, a target level for at least one hormone, and a target body mass index. In another embodiment, the RFM regimen is prescribed by a medical provider. In still another embodiment, the RFM regimen is based on rules stored in the memory. In one embodiment, the processor is included in a mobile device, such as a smart-phone or a tablet computer.

Optionally, the system can determine an adjustment to an insurance deductible for the patient based on the patient's progress in the RFM regimen. In one embodiment, the system can decrease the patient's insurance deductible when the patient has completed the RFM regimen. Alternatively, in another embodiment, the system can increase the patient's insurance deductible when the patient has not completed the RFM regimen.

Additionally, or alternatively, the system can determine an adjustment to an amount to be paid to a medical provider for the surgery based on the patient's progress in the RFM regimen. In one embodiment, the system can decrease the amount to be paid when the patient has not completed the RFM regimen. Alternatively, in another embodiment, the system can increase the amount to be paid when the patient has completed the RFM regimen.

In one embodiment, determining when the patient is ready for surgery comprises evaluating data in the medical record based on rules stored in the memory. Optionally, the system further comprises a rules module configured to determine when the patient is ready for surgery by evaluating data in the medical record in view of rules stored in the memory. Accordingly, determining when the patient is ready for surgery may comprise the rules module evaluating the data from the data monitor and determining that all requirements of the RFM regimen have been completed by the patient. Optionally, the system may further include altering a rule stored in the memory based on the post-surgery patient status report, wherein the rule is related to the RFM regimen.

In one embodiment, the system further comprises generating a report for a user. The report may comprise a user-interface to be displayed by a computer. In one embodiment, the user is the patient, a medical provider, and an insurance provider. In another embodiment, the report can be downloaded to a smartphone. Optionally, the report includes one ore more of an exercise program, a diet regimen, and a medication regimen for the patient. In one embodiment, the report includes information related to the patient's progress in the RFM regimen. The report can also include an estimated deductible to be paid by the patient for surgery based on the patient's progress in the RFM regimen. Optionally, the report includes at least one selectable icon. The selectable icon may graphically indicate the patient's progress in the RFM regimen. In one embodiment, the system generates the report in response to a request received from a user comprising one of a patient, a medical provider, and an insurance provider. Optionally, the request comprises the user opening a user-interface.

It is still another aspect of the present invention to provide a system for monitoring a plurality of health parameters associated with a patient to improve a surgical outcome. The system generally comprises, but is not limited to, one or more of: (a) a data monitor for the patient; and (b) a control unit to receive data from the patient, wherein the patient requires a surgical procedure, wherein the control unit is configured to store a pre-surgery regimen prescribed for the patient, and wherein the control unit is further configured to provide a recommendation as to the patient's readiness for the surgical procedure. In one embodiment, the data monitor includes at least one of: (i) a patient blood pressure data measurement device; (ii) a patient weight data measurement device; (iii) a patient physical activity data monitoring device; (iv) a patient heart rate data monitor; (v) a patient blood alcohol data measurement device; and (vi) a patient nicotine presence data measurement device. In one embodiment, the data monitor is an implantable medical device. In another embodiment, the control unit is an implantable device. Optionally, the control unit is a mobile device such as a smart-phone or a tablet computer. In another embodiment, the control unit is a computer server accessible remotely through the internet.

The pre-surgery regimen can be prescribed by at least one of a medical provider and an insurance provider. In one embodiment, the pre-surgery regimen is generated by the control unit. In another embodiment, the control unit includes a rules module and rules stored in memory which are related to the pre-surgery regimen. Optionally, the control unit can changes the rules based on analysis of a surgical outcome of the patient. Accordingly, the control unit can alter a rule, add a rule, or a delete a rule based on a surgical outcome of the patient. In one embodiment, the control unit can emphasize a rule based on the surgical outcome.

In one embodiment, the control unit is further configured to receive data related to the patient after the surgical procedure has been performed. Optionally, the control unit generates a post-surgery patient status report. In another embodiment, the control unit determines if the results of the surgery were acceptable.

In another embodiment, the pre-surgery regimen includes at least one of: (1) a dietary regimen; (2) an exercise program; (3) a medication protocol or schedule; (4) a target blood pressure; (5) a target blood glucose level; (6) lipid management; (7) arrhythmia management; (8) a reduction in alcohol consumption (or a maximum daily amount of alcohol allowed); (9) a reduction in tobacco use (or a maximum daily amount of tobacco allowed); (10) a target level for at least one hormone (such as a thyroid hormone level, an insulin level); (11) a target weight; (12) a target body mass index; and (13) a target cholesterol level. Optionally, the control unit is configured to provide at least one of an exercise program, a medication regimen, and a meal plan for the patient. The exercise program may include a schedule of one or more exercises to be performed by the patient for a predetermined length of time. The meal plan can include a recommended diet including an amount of protein, and amount of fats, and an amount of calories to be consumed by the patient daily.

One aspect of the present invention is a method of improving the success of a surgical procedure for a patient. The method generally includes: (1) providing a health regimen for a patient who requires surgery, the health regimen designed to improve the patient's health and reduce risk associated with surgery; (2) storing, by a control unit, the health regimen provided for the patient; (3) receiving data from a data monitor related to the patient, the data monitor operable to measure at least one of: (i) blood pressure; (ii) weight; (iii) physical activity; (iv) heart rate data; (v) blood alcohol data; (vi) nicotine presence; (vii) glucose levels; (viii) hormone levels; and (ix) cholesterol levels; and (4) providing a summary as to the patient's readiness for the surgery. In one embodiment, the method includes providing at least one of a dietary regimen, an exercise program, and a medication regimen to the patient. The method may further include generating a report after the surgical procedure is performed on the patient. Optionally, the control unit can alter, add, or delete a rule related to the health regimen based on the report.

Still another aspect of the present invention is a non-transitory computer readable media having stored thereon instructions, which when executed by a processor of a management system, cause the processor to perform a method of monitoring a plurality of health parameters associated with a patient to improve a surgical outcome. The instructions include, but are not limited to, one or more of: (1) an instruction to store, by the management system, a health regimen for the patient, the health regimen designed to improve the patient's health and reduce risk associated with surgery; (2) an instruction to receive data from a data monitor related to the patient, the data monitor operable to measure at least one of: (i) blood pressure; (ii) weight; (iii) physical activity; (iv) heart rate data; (v) blood alcohol data; and (vi) nicotine presence; and (3) an instruction to determine when the patient is ready for surgery. Optionally, the instructions further include an instruction for the management system to review patient data and provide one or more of a dietary regimen, an exercise program, and a medication protocol for the patient. In another embodiment, the instructions include an instruction to generate a user interface for one or more of the patient and a medical provider, the user interface including information on the patient's progress in the pre-surgery regimen.

Although the RFM system is generally described in relation to surgery planned for the patient, one of skill in the art will appreciate that the RFM system and method of the present invention are not limited to patients scheduled for surgery. Accordingly, it is an aspect of the present invention to provide an RFM system to track progress of patients in a prescribed RFM regimen whether or not the patients require surgery.

The above-described embodiments, objectives, and configurations are neither complete nor exhaustive. As will be appreciated, other embodiments of the disclosure are possible using, alone or in combination, one or more of the features set forth above or described in detail below.

“The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.”

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refer to any process or operation done without material human input when the process or operation is performed. However, a process or operation can be automatic, even though performance of the process or operation uses material or immaterial human input, if the input is received before the performance of the process or operation. Human input is deemed to be material if such input influences how the process or operation will be performed. Human input that consents to the performance of the process or operation is not deemed to be “material.”

The terms “communication device,” “smartphone,” and “mobile device,” and variations thereof, as used herein, can be used interchangeably and may include any type of device capable of communicating with one or more of another device and/or across a communications network, via a communications protocol, and the like. Exemplary communication devices may include but are not limited to smartphones, handheld computers, laptops, netbooks, notebook computers, subnotebooks, tablet computers, scanners, portable gaming devices, phones, pagers, GPS modules, portable music players, and other Internet-enabled and/or network-connected devices.

A “communication modality” can refer to any protocol or standard defined or specific communication session or interaction, such as Voice-Over-Internet-Protocol (“VoIP), cellular communications (e.g., IS-95, 1G, 2G, 3G, 3.5G, 4G, 4G/IMT-Advanced standards, 3GPP, WIMAX™, GSM, CDMA, CDMA2000, EDGE, 1×EVDO, iDEN, GPRS, HSPDA, TDMA, UMA, UMTS, ITU-R, and 5G), Bluetooth™, text or instant messaging (e.g., AIM, Blauk, eBuddy, Gadu-Gadu, IBM Lotus Sametime, ICQ, iMessage, IMVU, Lync, MXit, Paltalk, Skype, Tencent QQ, Windows Live Messenger™ or MSN Messenger™, Wireclub, Xfire, and Yahoo! Messenger™), email, Twitter (e.g., tweeting), Digital Service Protocol (DSP), and the like.

The term “communication system” or “communication network” and variations thereof, as used herein, can refer to a collection of communication components capable of one or more of transmission, relay, interconnect, control, or otherwise manipulate information or data from at least one transmitter to at least one receiver. As such, the communication may include a range of systems supporting point-to-point or broadcasting of the information or data. A communication system may refer to the collection individual communication hardware as well as the interconnects associated with and connecting the individual communication hardware. Communication hardware may refer to dedicated communication hardware or may refer a processor coupled with a communication means (i.e., an antenna) and running software capable of using the communication means to send and/or receive a signal within the communication system. Interconnect refers some type of wired or wireless communication link that connects various components, such as communication hardware, within a communication system. A communication network may refer to a specific setup of a communication system with the collection of individual communication hardware and interconnects having some definable network topography. A communication network may include wired and/or wireless network having a pre-set to an ad hoc network structure.

The term “computer-readable medium,” as used herein refers to any tangible storage and/or transmission medium that participates in providing instructions to a processor for execution. Such a medium may take many forms, including but not limited to, non-volatile media, volatile media, and transmission media. Non-volatile media includes, for example, non-volatile random access memory (NVRAM), or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a compact disc read only memory (CD-ROM), any other optical medium, punch cards, paper tape, any other physical medium with patterns of holes, a random access memory (RAM), a programmable read only memory (PROM), and erasable programmable read only memory EPROM, a FLASH-EPROM, a solid state medium like a memory card, any other memory chip or cartridge, a carrier wave as described hereinafter, or any other medium from which a computer can read. A digital file attachment to an e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. It should be noted that any computer readable medium that is not a signal transmission may be considered non-transitory.

The term “module” as used herein refers to any known or later developed hardware, software, firmware, artificial intelligence, fuzzy logic, or combination of hardware and software that is capable of performing the functionality associated with that element.

The terms “determine,” “calculate,” and “compute,” and variations thereof, as used herein, are used interchangeably and include any type of methodology, process, mathematical operation, or technique.

The term “in communication with,” as used herein, refers to any coupling, connection, or interaction using electrical signals to exchange information or data, using any system, hardware, software, protocol, or format, regardless of whether the exchange occurs wirelessly or over a wired connection.

The term “Bluetooth” may refer to wireless technology for exchanging data over short distances (using short-wavelength UHF radio waves in the ISM band) from fixed and mobile devices and building personal area networks (PANs). The technology may connect several devices in order for data synchronization between devices or between devices and a server.

It shall be understood that the term “means,” as used herein, shall be given its broadest possible interpretation in accordance with 35 U.S.C., Section 112, Paragraph 6 or other applicable law. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The Summary of the Invention is neither intended, nor should it be construed, as being representative of the full extent and scope of the present invention. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description and no limitation as to the scope of the present invention is intended by either the inclusion or non-inclusion of elements or components. Additional aspects of the present invention will become more readily apparent from the Detailed Description, particularly when taken together with the drawings.

### BRIEF DESCRIPTION OF THE DRAWINGS

The accompanying drawings, which are incorporated herein and constitute a part of the specification, illustrate embodiments of the invention and together with the Summary of the Invention given above and the Detailed Description given below serve to explain the principles of these embodiments. In certain instances, details that are not necessary for an understanding of the disclosure or that render other details difficult to perceive may have been omitted. It should be understood, of course, that the present invention is not necessarily limited to the particular embodiments illustrated herein. Additionally, it should be understood that the drawings are not necessarily to scale.

FIG. 1 is a block diagram illustrating elements of an exemplary computing environment in which embodiments of the present disclosure may be implemented;

FIG. 2 is a block diagram illustrating elements of an exemplary computing device in which embodiments of the present disclosure may be implemented;

FIG. 3 is a block diagram of an embodiment of a risk factor modification system of one embodiment of the present invention;

FIG. 4 is a schematic view of one embodiment of a patient interface produced by the RFM system of FIG. 3;

FIG. 5 illustrates one embodiment of a user interface for a medical provider produced by the RFM system of FIG. 3;

FIG. 6 is graphical representation of a user interface produced by the RFM system of FIG. 3 that is provided for a medical administrator;

FIG. 7 is a flow or process diagram of a method generally illustrating pre-surgery patient data flow for determining if the patient has satisfied a prescribed pre-surgery RFM regimen;

FIG. 8 is another flow or process diagram illustrating a method of post-surgery patient data flow;

FIG. 9 generally illustrates another flow or process diagram for a medical provider to determine when a patient is ready for surgery;

FIG. 10 generally illustrates another flow or process diagram for a medical provider or an insurance provider to determine when a patient is ready for surgery;

FIG. 11 is another flow or process diagram that generally illustrates a method of determining whether a patient is ready for surgery based on criteria established by a pre-surgery RFM regimen;

FIG. 12 illustrates still another flow or process diagram for surgery approval by an insurance provider using the system of FIG. 3; and

FIG. 13 is another flow or process diagram of determining whether an outcome of surgery performed on a patient meets established expectations of care.

To assist in the understanding the present disclosure, a list of components and associated numbering found in the drawings is provided herein:

Number Component

100 Computing environment

104 Computing device

108 Computing device

110 Network

112 Communication device

114 Server

116 Server

118 Database

200 Computer system

204 Bus

208 CPU

212 Input devices

216 Output devices

220 Storage devices

224 Computer-readable storage media reader

228 Communication system

232 Processing acceleration unit

236 Working memory

240 Operating system

244 Other code

300 RFM system

302 Storage device

304 Service provider system

308 Management system

312 Rules module

316 Rules database

320 Data collection module

324 Records database

328 Patient

332 Patient system

336 Sensors

336A Heart monitor

336B Activity monitor

336C Temperature monitor

336D Blood monitor

336E Weight monitor

336F Other sensors

340 Heart/respiration monitor

344 Smart watch/fitness monitor

400 Patient user-interface

404 Display portion

408 Current status

412 Target

416 Scroll bar

420 Field

424 Progress display area

428 Suggestions icon

432 Chat icon

500 Medical provider interface

504 Display area

508 Status icon

512 Detail icon

516 Scrollbar

520 Chat icon

524 Office visit icon

528 Schedule surgery icon

532 Link button

536 Link button

540 Sort button

544 Option button

600 Medical administrator interface

604 First display area

608 Status column

612 Insurance column

616 At risk column

620 Second display area

624 Scroll bar

628A First report icon

628B Second report icon

628C Third report icon

628D Fourth report icon

640 Sort button

644 Option button

700 Method

704 Start

708 Establish patient RFM regimen

712 Measure/record heart rate

716 Measure/record blood pressure

720 Measure/record weight

724 Measure/record activity

728 Measure/record other data

732 Determine if RFM regimen completed

736 Update patient status to ready

740 Update patient status to not ready

744 End

800 Method

812 Measure/record heart rate

816 Measure/record blood pressure

820 Measure/record weight

824 Measure/record activity

828 Measure/record other data

832 Check patient status

836 Update post-operative outcome data

840 End

900 Method

904 Start

908 Receive data request

912 Retrieve patient data

914 Check patient surgery status

916 Determine if patient is ready for surgery

920 Patient not ready

924 Patient ready for surgery

928 End

1000 Method

1004 Start

1008 Receive data request

1012 Retrieve patient data

1014 Check patient surgery status

1016 Determine if patient is ready for surgery

1020 Patient not ready

1024 Patient ready for surgery

1028 End

1100 Method

1104 Start

1108 Establish an RFM regimen

1112 Determine whether weight loss is satisfied

1116 Determine if blood pressure requirement is satisfied

1120 Determine if alcohol and tobacco use requirements are satisfied

1124 Determine if sleep apnea has been treated

1128 Determine if exercise requirement is satisfied

1132 Determine if medication requirement is satisfied

1136 Determine if other requirements have been satisfied

1140 Update patient status

1144 End

1200 Method

1204 Start

1208 Receive data request

1212 Retrieve patient data

1214 Check patient surgery status

1216 Determine if patient is ready for surgery

1220 Patient not ready

1224 Patient ready for surgery

1228 Insurance provider approve surgery

1232 End

1300 Method

1304 Start

1308 Receive data request

1312 Retrieve patient data

1314 Check patient surgery outcome status

1316 Determine if outcome time period is satisfied

1320 Determine whether outcome is within tolerance

1324 Generate report

1328 Request refund

1332 End

### DETAILED DESCRIPTION

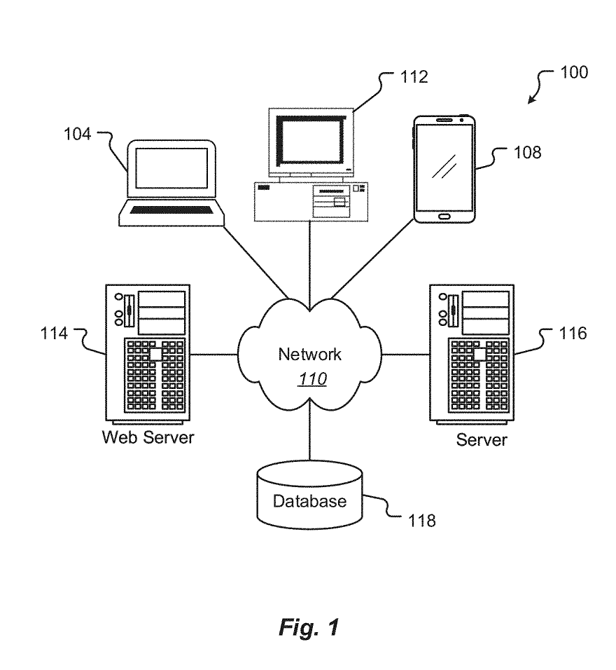

Referring now to FIG. 1, a block diagram is provided illustrating elements of an exemplary computing environment in which embodiments of the present disclosure may be implemented. More specifically, this example illustrates a computing environment 100 that may function as the servers, user computers, or other systems provided and described herein. The environment 100 includes one or more user computers, or computing devices, such as a computing device 104, a communication device 108, and/or more computing devices 112. The computing devices 104, 108, 112 may include general purpose personal computers (including, merely by way of example, personal computers, and/or laptop computers running various versions of Microsoft Corp.'s Windows® and/or Apple Corp.'s Macintosh® operating systems) and/or workstation computers running any of a variety of commercially-available UNIX® or UNIX-like operating systems. These computing devices 104, 108, 112 may also have any of a variety of applications, including for example, database client and/or server applications, and web browser applications. Alternatively, the computing devices 104, 108, 112 may be any other electronic device, such as a thin-client computer, Internet-enabled mobile telephone, and/or personal digital assistant, capable of communicating via a network 110 and/or displaying and navigating web pages or other types of electronic documents. Although the exemplary computer environment 100 is shown with three computing devices 104, 108, 112, any number of user computers or computing devices may be supported.

Environment 100 further includes a network 110. The network 110 may can be any type of network familiar to those skilled in the art that can support data communications using any of a variety of commercially-available protocols, including without limitation SIP, TCP/IP, SNA, IPX, AppleTalk, and the like. Merely by way of example, the network 110 maybe a local area network (“LAN”), such as an Ethernet network, a Token-Ring network and/or the like; a wide-area network; a virtual network, including without limitation a virtual private network (“VPN”); the Internet; an intranet; an extranet; a public switched telephone network (“PSTN”); an infra-red network; a wireless network (e.g., a network operating under any of the IEEE 802.9 suite of protocols, the Bluetooth® protocol known in the art, and/or any other wireless protocol); and/or any combination of these and/or other networks.

The system may also include one or more servers 114, 116. In this example, server 114 is shown as a web server and server 116 is shown as an application server. The web server 114, which may be used to process requests for web pages or other electronic documents from computing devices 104, 108, 112. The web server 114 can be running an operating system including any of those discussed above, as well as any commercially-available server operating systems. The web server 114 can also run a variety of server applications, including SIP (Session Initiation Protocol) servers, HTTP(s) servers, FTP servers, CGI servers, database servers, Java servers, and the like. In some instances, the web server 114 may publish operations available operations as one or more web services.

The environment 100 may also include one or more file and or/application servers 116, which can, in addition to an operating system, include one or more applications accessible by a client running on one or more of the computing devices 104, 108, 112. The server(s) 116 and/or 114 may be one or more general purpose computers capable of executing programs or scripts in response to the computing devices 104, 108, 112. As one example, the server 116, 114 may execute one or more web applications. The web application may be implemented as one or more scripts or programs written in any programming language, such as Java™, C, C#®, or C++, and/or any scripting language, such as Perl, Python, or TCL, as well as combinations of any programming/scripting languages. The application server(s) 116 may also include database servers, including without limitation those commercially available from Oracle®, Microsoft®, Sybase®, IBM® and the like, which can process requests from database clients running on a computing device 104, 108, 112.

The web pages created by the server 114 and/or 116 may be forwarded to a computing device 104, 108, 112 via a web (file) server 114, 116. Similarly, the web server 114 may be able to receive web page requests, web services invocations, and/or input data from a computing device 104, 108, 112 (e.g., a user computer, etc.) and can forward the web page requests and/or input data to the web (application) server 116. In further embodiments, the server 116 may function as a file server. Although for ease of description, FIG. 1 illustrates a separate web server 114 and file/application server 116, those skilled in the art will recognize that the functions described with respect to servers 114, 116 may be performed by a single server and/or a plurality of specialized servers, depending on implementation-specific needs and parameters. The computer systems 104, 108, 112, web (file) server 114 and/or web (application) server 116 may function as the system, devices, or components described herein.

The environment 100 may also include a database 118. The database 118 may reside in a variety of locations. By way of example, database 118 may reside on a storage medium local to (and/or resident in) one or more of the computers 104, 108, 112, 114, 116. Alternatively, it may be remote from any or all of the computers 104, 108, 112, 114, 116, and in communication (e.g., via the network 110) with one or more of these. The database 118 may reside in a storage-area network (“SAN”) familiar to those skilled in the art. Similarly, any necessary files for performing the functions attributed to the computers 104, 108, 112, 114, 116 may be stored locally on the respective computer and/or remotely, as appropriate. The database 118 may be a relational database, such as Oracle 20i®, that is adapted to store, update, and retrieve data in response to SQL-formatted commands.

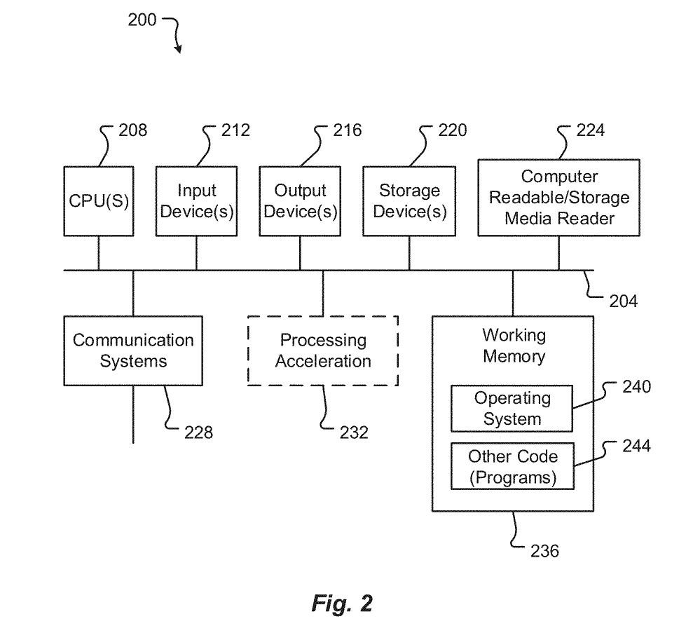

Referring now to FIG. 2, another block diagram is provided which illustrates elements of an exemplary computing device in which embodiments of the present disclosure may be implemented. More specifically, this example illustrates one embodiment of a computer system 200 upon which the servers, user computers, computing devices, or other systems or components described above may be deployed or executed. The computer system 200 is shown comprising hardware elements that may be electrically coupled via a bus 204. The hardware elements may include one or more central processing units (CPUs) 208; one or more input devices 212 (e.g., a mouse, a keyboard, etc.); and one or more output devices 216 (e.g., a display device, a printer, etc.). The computer system 200 may also include one or more storage devices 220. The storage device(s) 220 may be disk drives, optical storage devices, solid-state storage devices such as a random-access memory (“RAM”) and/or a read-only memory (“ROM”), which can be programmable, flash-updateable and/or the like.

The computer system 200 may additionally include a computer-readable storage media reader 224; a communications system 228 (e.g., a modem, a network card (wireless or wired), an infra-red communication device, etc.); and working memory 236, which may include RAM and ROM devices as described above. The computer system 200 may also include a processing acceleration unit 232, which can include a DSP, a special-purpose processor, and/or the like.

The computer-readable storage media reader 224 can further be connected to a computer-readable storage medium, together (and, optionally, in combination with storage device(s) 220) comprehensively representing remote, local, fixed, and/or removable storage devices plus storage media for temporarily and/or more permanently containing computer-readable information. The communications system 228 may permit data to be exchanged with a network and/or any other computer described above with respect to the computer environments described herein. Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine-readable mediums for storing information.

The computer system 200 may also comprise software elements, shown as being currently located within a working memory 236, including an operating system 240 and/or other code 244. It should be appreciated that alternate embodiments of a computer system 200 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 208 as described herein may include, but are not limited to, at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-STM processors, other industry-equivalent processors, and may perform computational functions using any known or future-developed standard, instruction set, libraries, and/or architecture.

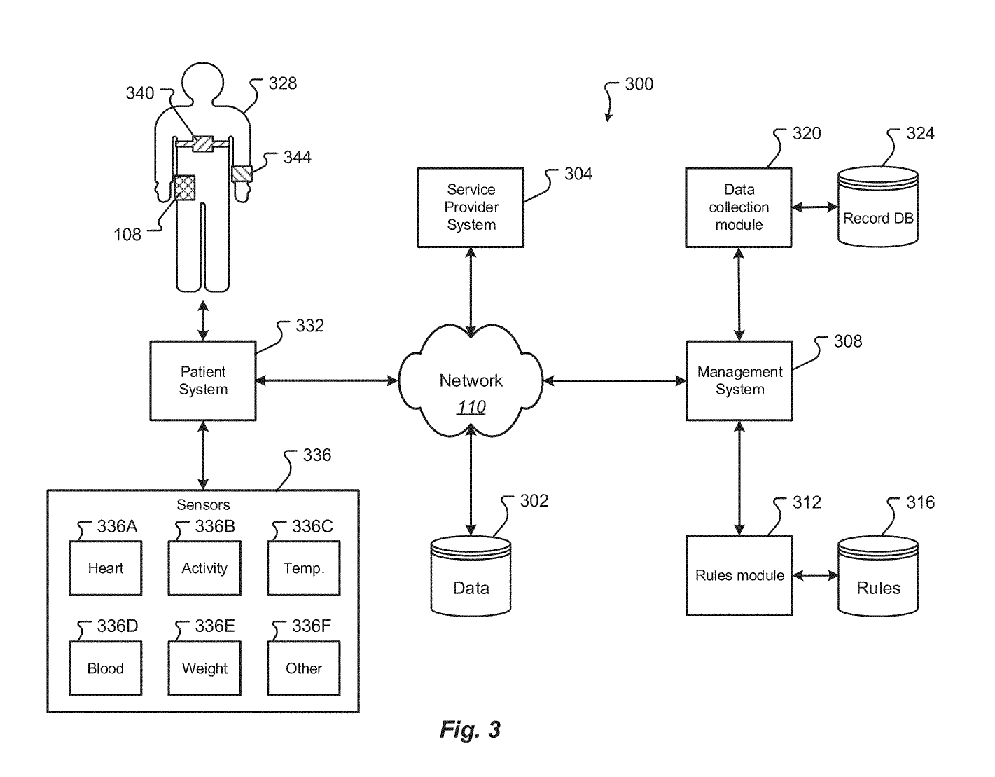

Referring now to FIG. 3, a block diagram illustrating an exemplary RFM system 300 of one embodiment of the present invention is generally illustrated. The RFM system 300 may comprise hardware and/or software that conduct various operations for different users. The users may include, but are not limited to, a patient, a medical provider, a medical administrator, and an insurance provider. The operations can include providing information to a user, collecting data associated with a user, receiving input from a user, and providing recommendations and alerts to a user.

The RFM system 300 generally includes a service provider system 304, a management system 308, and a patient system 332. The systems 304, 308, 332 can be communicatively coupled with a communication network 110 such as the Internet or any other one or more wired or wireless, local or wide area networks. The communication network 110 can include a local area communication capability and a wide area communication capability. For example, the communication network 110 can include a Bluetooth® wireless system, an 802.11x (e.g., 802.11G/802.11N/802.11AC, or the like, wireless system), a CAN bus, an Ethernet network connecting systems 304, 308, 332, or other types of communication networks that may function with or be associated with the modification system of the present invention. Further, the communication network 110 can also include wide area communication capabilities, including one or more of, but not limited to, a cellular communication capability, satellite telephone communication capability, a wireless wide area network communication capability, or other types of communication capabilities that allow elements of the RFM system 300 to communicate.

The systems 304, 308, 332 may also be operable to communicate with other devices using communicate network 110, including remote storage devices 302. In one embodiment, one or more of the patient system 332 and the service provider system 304 may be a mobile device such as a smart phone, a mobile computer, a smart watch, or other type of mobile computing system or device.

The RFM system 300 can include any number of service provider systems 304. Each service provider system 304 can comprise a server or other computing device as described above. The service provider system 304 is associated with a user such as one or more of a healthcare provider (a hospital, a doctor, physical therapists, medical administrators, counsellors, out-patient and/or urgent care facilities, pharmacies, or other such providers) and responsible parties such as third-party payors including but not limited to insurance companies, Medicare, Medicaid, and/or other private, governmental, or mixed public/private entities.

The management system 308 is operable to manage patient records, including updating patient records with data related to a RFM regimen, collect data, and retrieve patient records and data in response to an input from a user. The management system 308 can be can be any type of computing system operable to conduct the operations as described herein. Optionally, the management system 308 is a computing device 114, 116, 200 such as described above. Accordingly, the management system 308 can include a processor 208 and memory 220, 236.

The RFM system 300 can maintain a plurality of records related to medical services provided to a patient 328 by a plurality of medical providers. Accordingly, the management system 308 can retrieve records, record biometric data received from a patient 328 or other user, update a record to indicate a patient is ready for surgery, update the record to indicate surgery has been approved for a patient by a doctor and/or by an insurer, and record data on a patient after surgery has been performed. Specifically, the management system 308 can maintain records related to a RFM regimen prescribed for a patient. The management system 308 can also update a record of a patient to indicate the patient has been approved for aa medical procedure by a medical provider and/or an insurance provider.

The management system 308 may be located in a facility that is not within physical proximity to the patient system 332 or the service provider system 304. The management system 308 may include, or have access to, storage systems 302, 316, 324 to store system data. The system data may be any type of data needed for operation of the RFM system 300. Optionally, the system data may represent any type of database or other storage system. The system data can be stored in a cloud or in a distant facility, may be exchanged among the patient system 332, the service provider system 304, and the management system 308, and can be used by users in different locations or terminals that connect to the management system 308. Thus, the management system 308 may represent a cloud computing system or cloud storage that allows patient system 332 and/or the service provider system 304 to either gain access to further computing capabilities or to storage 302, 316, 324 at a location distant to the patient system and the service provider system.

The management system 308 can store patient records related to a pre-surgery RFM regimen for a patient 328 in a database 324. In one embodiment, an RFM regimen can be established by a medical provider using a service provider system 304. Optionally, the RFM regimen is specific to surgery planned for a patient 328. The RFM regimen can be customized for the patient 328.

Additionally, or alternatively, in one embodiment the management system 308 can automatically generate an RFM regimen for a patient. For example, in one embodiment, the management system 308 includes a rules module 312 which applies rules stored in memory 316. The rules may describe one or more elements of an RFM regimen. In one example, a rule may include abstinence from alcohol and/or tobacco for a predetermined period of time. Another rule may be associated with a predetermined amount of weight loss, either in pounds or as a percentage of the patient's starting weight. Other rules can define an acceptable or desired blood pressure, treatment of a disorder (such as sleep apnea), required exercise, and compliance with medication requirements. Another rule may be associated with a patient's AFib burden or hypertension.

The rules may be associated with a variety of risk factors and biometric factors for surgical procedures. In one embodiment, the rules can define one or more of: a weight, a body mass index, a blood pressure, a heart rate, a heart rhythm, a temperature, a respiration rate, a lipid level, a glucose level, a permissible alcohol use amount, and a permissible tobacco use amount. The rules may also include sleep-disordered breathing management. Some risk factors may be weighted differently than other risk factors. For example, the rules may assign a greater weight, or emphasis to a first risk factor and a lesser weight, or deemphasize, a second risk factor. The rules may be based on the age and sex of the patient. The rules database 316 can include records related to surgical outcomes for a plurality of patients. More specifically, the rules database may include information for a plurality of patients and results of surgery performed on the patients. The information may include successful outcomes, unsuccessful outcomes, and an RFM regimen prescribed for the patient.

The rules module can compare patient information to information in the rules database 316 to generate a predicted outcome for the patient. More specifically, based on the patient's age, sex, health data, and a planned surgery, the rules module 312 can provide a predicted outcome of a surgical procedure planned for the patient based on outcomes of similar surgeries performed on other patients. The predicted outcome may be expressed as a percent chance of achieving a successful surgery. Optionally, the rules module 312 can determine one or more risk factors, such as blood pressure, weight, heart rate, exercise level, alcohol consumption, and tobacco use, that the patient should change to improve the chance of achieve a successful surgical outcome. In this manner, the rules module can use patient data to predict the results of a surgical procedure based on results of similar surgical procedures performed on a plurality of patients.

Rules may also be associated with one or more of: a type of atrial fibrillation (AF) (including paroxysmal AF and nonparoxysmal AF); metabolic risk factors; medications; echocardiographic measures, and atrial fibrillation severity. Optionally, the rules may be associated with one or more surgical procedures. In one embodiment, the rules are associated with catheter ablation.

The rules may be associated with metabolic risk factors including, but not limited to: hypertension, diabetes mellitus, hyperlipidemia, coronary artery disease, an apnea-hypopnea index (AHI), alcohol consumption greater than 30 g/week, and tobacco use. The echocardiographic measures may include rules related to left atrial volume index, left ventricular septum, left ventricular internal dimension in diastole, and left ventricular ejection fraction. Optionally, one or more rules may be associated with a atrial fibrillation severity scale including frequency, duration, severity, symptom, and global well-being.

The management system 308 can use the rules in database 316 to create an RFM regimen for a patient 328. The rules module 312 can use patient data received from a patient system 332 or input by a medical provider using service provider system 304 to determine risk factors for the patient that should be modified prior to a planned surgical procedure. The risk factors may be determined at least in part based on the rules in database 316. With this information, the rules module 312 can generate an RFM regimen for a patient.

The RFM regimen may include one or more of, but is not limited to: a target weight, a target body mass index, a target blood pressure, a target heart rate, a target respiration rate, a target lipid level, a target glucose level, a permissible alcohol use amount, and a permissible tobacco use amount. The RFM regimen may include one or more of dietary instructions, exercise instructions, and medication instructions. Optionally, a medical provider may create the RFM regimen using the rules. In another embodiment, the medical provider can alter the RFM regimen generated by the RFM system 300.

In one embodiment, medical literature is accessible to the management system 308. For example, in one embodiment, the RFM system 300 can access medical literature stored in a remote database 302. Optionally, medical literature may also be stored in database 316. The medical literature can be used by the rules module 312 to create new rules or update existing rules stored in database 316.

Optionally, the rules module 312 can create or update rules based on surgical outcomes of patients. In one embodiment, the surgical outcomes are compared to RFM regimens prescribed for patients. In this manner, the rules module 312 can improve operation of the RFM system 300. For example, the rules module 312 may increase the weight or importance of an element of a RFM regimen that is linked to an improved surgical result. Similarly, the rules module 312 may deemphasize, or eliminate, an element of a RFM regimen that is determined to be less important to improved surgical results. In one embodiment, the rules module 312 compares surgical outcomes of patients to their compliance with a RFM regimen. The rules module 312 then determines if one or more elements of a RFM regimen are statistically relevant to a successful, or adverse, surgical procedure.

After a RFM regimen is established for a patient, the management system 308 may receive data from a patient system 332 related to elements of the patient's RFM regiment. The RFM system 300 may include a data collection module 320 to maintain patient records in a database 324. The data collection module 320 can receive data from a patient system 332 and update a patients record in the database 324. Specifically, the data collection module 320 of the management system 308 processes data received from the patient system 332 and the service provider system 304. The data collection module can store the data received from systems 304, 332 in database 324 in a record structure that is defined by a RFM regimen. RFM regimens may be stored in the management system 308 for any number of patients 328. By comparing the collected patient data received from the patient system 332 to the rules associated with a patient's prescribed RFM regimen, the RFM system 300 can determine whether the patient has complied with each of the elements of the pre-surgery RFM regimen.

The patient system 332 may be associated with a user 328, such as a patient. Although only one patient system 332 is illustrated, one of skill in the art will appreciate that the RFM system 300 may include any number of patient systems 332 associated with a plurality of patients 328.

The patient system 332 can receive data related to the patient 328 from sensors 336. The sensors 336 may be configured to provide information to the patient system 332 related to a RFM regimen prescribed for the patient. The sensors 336 may be integral to the patient system 332. Additionally, or alternatively, external sensors may also collect biometric data of the patient 328 and communicate the data over the network 110 to the management system 308. The sensors 336 are operable to collect any type of biometric data of the user. The data collected by the sensors 336 may include, but is not limited to, at least one of weight, blood pressure, heart rate, heart rhythm (including when the patient is experiencing atrial fibrillation), blood or breath alcohol level, blood chemistry (such as to measure at least one of alcohol, sodium, oxygen, carbon dioxide, glucose, and insulin levels), respiration rate, temperature, and activity (such as a number of steps taken by the patient). Optionally, a sensor 336 can measure one or more hormone levels of the patient. For example, a sensor may measure a thyroid level or an insulin level. In one embodiment, one or more of the sensors may be implantable in the patient.

The sensors 336 can include one or more of a heart monitor 336A, an activity monitor 336B, a temperature monitor 336C, a blood monitor 336D, a weight monitor 336E, and others 336F, which are in communication with and provide patient data to the patient system 332. In one embodiment, the heart monitor 336A can measure one or more of a heart rate and a heart rhythm of a patient. Optionally, one or more of the sensors 336 may be associated with a wearable device, such as a heart/respiration monitor 340, a smart watch or fitness monitor 344, a smart phone 108, and the like.

The sensors 336 may include sensor memory. Embodiments of the sensor memory may be configured to store data collected by the sensors 336 for any length of time. The sensors 336 may collect and transmit data on a schedule determined by rules stored in memory 316 of the management system 308. For example, the management system 308 may specify when the patient system 332, and its associated sensors 336, should collect and/or transmit data to the management system. Thus, the sensors 336 may periodically collect data of the patient 328. The data may be collected incrementally, in response to a condition, or at specific time periods. In this example, as the data is collected, it may be stored in the sensor memory. In some cases, the data may be stored along with an identification of the sensor and a collection time associated with the data. Among other things, this stored data may include multiple data points and may be used to track changes in sensor measurements over time. As can be appreciated, the sensor memory can represent any type of database or other storage system.

The patient system 332 may also be operable to receive data input from the patient 328. For example, the patient 328 may enter data into the patient system 332 related to compliance with one or more activities prescribed as part of a RFM regimen. The patient may enter the data using an input device 212 of the patient system 332.

The patient system 332 may also receive alerts, reports, and user-interfaces from the management system 308. In one example, management system 308 processes the data received from the patient system 332. The management system 308 may then determine whether or not the patient 328 has completed the patient's prescribed RFM regimen. In response, the management system 308 may generate an alert or report that is provided to the patient 328 by the patient system 332. The management system 308 may also send the alert or report to the service provider system 304. The report may include a summary of the patient's progress in the RFM regimen. One example is a report indicting the patient 328 is ready for surgery. Optionally, the alert may include suggested activities to be performed by the patient 328 to comply with the RFM regimen.

The management system 308 can generate reports and/or user interfaces to a patient system 332 or a service provider system 304 operated by the users based on their secure access permissions. These reports assess the patient's readiness in the context of established pre-surgery RFM regimen that produce optimal surgical outcomes for a given clinical condition. As previously described, the reports may be specific to different users. A patient user may thus receive reports with different information and formats than the other users. Medical providers and medical administrators may receive reports indicating that their patients are ready, or are not ready, for surgery. Similarly, insurance providers, both private and public (including the Centers for Medicare and Medicaid Services (CMS)) can receive reports that indicate the status of patients they insure in a prescribed RFM regimen. All users may also receive patient follow up outcome reports at one or more times after surgery.

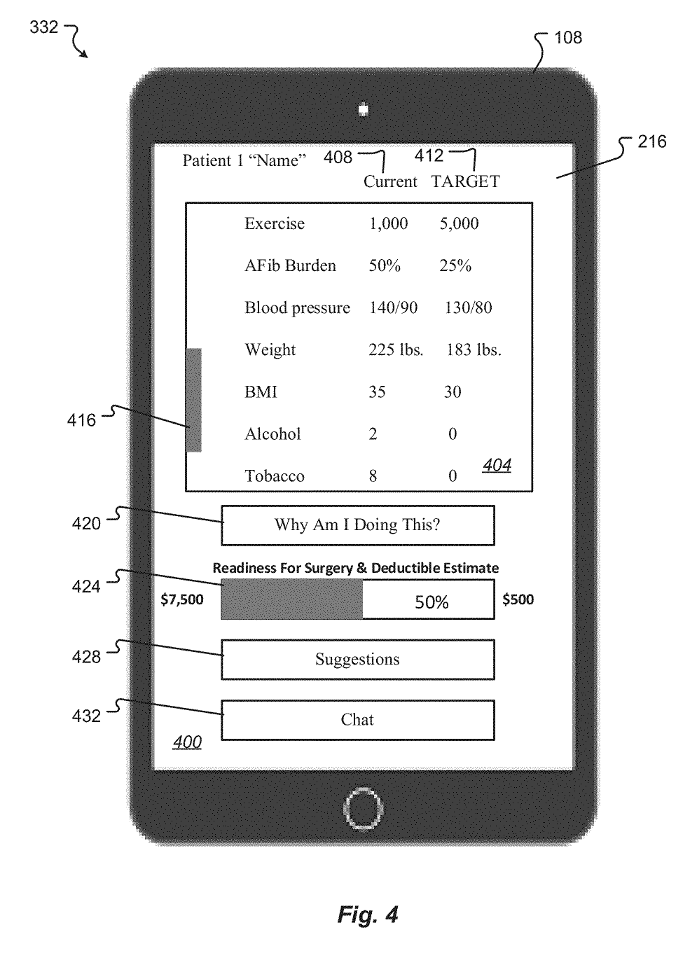

Referring now to FIG. 4, an example of a patient user-interface 400 of one embodiment of the present invention is provided. The patient user-interface 400 may be generated by the management system 308 and transmitted to the patient system 332. Additionally, the patient interface can be presented by a display 216 of the patient system 332. In one embodiment, the patient user-interface 400 may be presented by a smart device 108 operated by a patient 328.

Through the patient user-interface 400, the patient 328 can monitor their progress in the context of the pre-surgery RFM regimen that they must follow and risk factor criteria they must achieve to have the best possible surgical outcome. Thus, the patient user-interface 400 provides information to the user relevant to the patient's readiness for surgery. The patient user-interface 400 provides constant feedback and suggestions to adhere and improve the patient's adherence to a prescribed RFM regimen.

The patient user-interface 400 includes a display portion 404 which presents one or more elements of the patient's RFM regimen. The display portion 404 may include information related to the patient's current status 408 and targets 412 for one or more elements of the RFM regimen. For example, a daily level and a target level for each element of the RFM regimen may be displayed. The display portion 404 can include a selectable device 416, such as a scroll bar, to display more, or different, information.

In one embodiment, the display portion 404 can provide information on one or more elements of the patients RFM regimen. For example, an amount of physical activity of the patient can be displayed. In one embodiment, the physical activity is an amount of aerobic activity of the patient, such as a number of steps during a day.

The patient's AFib burden may also be displayed. The AFib burden can be displayed as a percentage. As will be appreciated by one of skill in the art, the AFib burden describes the percent of time spent by the patient in atrial fibrillation.

The display portion 404 can also include information associated with one or more of blood pressure, weight, body mass index (“BMI”), alcohol consumption, and tobacco consumption. Other fields are contemplated and may be displayed by moving the scroll bar 416. Specifically, the display portion 404 can include information on any element of a RFM regimen prescribed for the patient.

The patient user-interface 400 also can provide information as to why this RFM regimen is important to the success of the surgery. For example, in one embodiment, the patient user-interface 400 includes a field 420 which can be selected by the patient. The field 420 can provide information to the patient related to the benefits of the RFM regimen. For example, if the patient selects the field 420, the patient system 332 can retrieve data from a database 302, 316 which describes the benefit of one or more aspects of the RFM regimen prescribed to the patient.

The patient user-interface 400 may also provide potential financial implications based on the status of the patient in a progress display area 424. Specifically, the progress display area 424 can present information such as the patient's progress in the RFM regimen. The progress may be presented as a percentage of completion of the RFM regimen. In one embodiment, the progress display area 424 present information generated by one or more of methods 700 to 1300 described herein.

Additionally, or alternatively, the progress display area 424 can also display a financial incentive associated with the patient's progress in the pre-surgery regimen. The financial incentive may comprise a reduction in a co-pay or deductible provided by the patient's insurance carrier. In this manner, the insurance carrier may waive, or decrease the amount the patient must pay for a surgical procedure. Thus, the patient is given an incentive to complete, or at least make positive progress in, the RFM regimen. In one example, as the patient progresses toward target values set for elements of the RFM regimen, the amount of the deductible paid by the patient decreases.

Also, the patient user-interface 400 may include a mechanism for the patient to reach a community of help in the form of coaching and communication. Accordingly, in one embodiment, the patient user-interface 400 includes one or more of a suggestions icon 428 and a chat icon 432. If the patient selects the chat icon 432, the patient system 332 can connect the patient to a medical provider such as a doctor, a physical therapist, a counsellor, or other such providers.

When the suggestions icon 428 is selected, the patient system 332 can connect to the management system 308. The management system 308 can retrieve the patient's record from database 324. The management system 308 can then review the patient's progress in the RFM regimen and compare the patient's progress to rules stored in database 316. Optionally, the management system 308 can access data stored in database 302. In one embodiment, the management system 308 can provide results of surgical outcomes for similar patients that have completed an RFM regimen to the patient. Regardless, the management system 308 can present information to the patient such as portions of the RFM regimen the patient should focus on, actions the patient can take to progress in the RFM regimen, benefits of the RFM regimen, and individuals that patient can contact for help completing the RFM regimen.

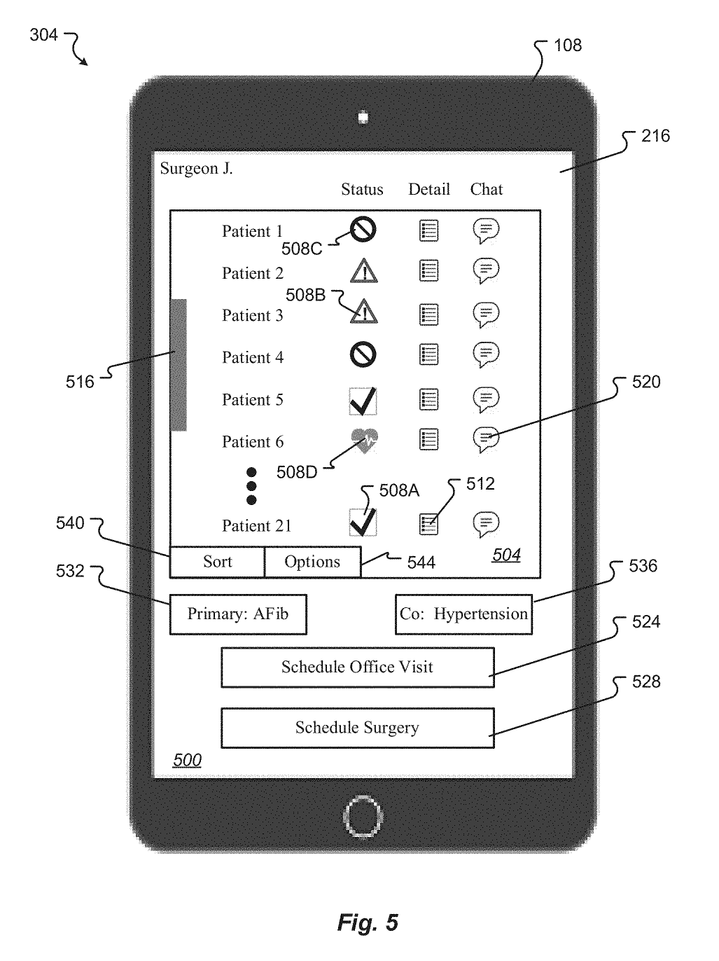

Referring now to FIG. 5, a medical provider interface 500 of one embodiment of the present invention is generally illustrated. Through the medical provider interface 500, a user, such as a surgeon, can monitor the progress of patients in RFM regimens when desired, including substantially constantly. The surgeon can assess each patient's readiness for surgery. The surgeon can further examine the details of each patient's progress along a prescribed RFM regimen and modify the RFM regimen as appropriate. The surgeon can use the medical provider interface 500 to schedule the patient for an office visit and/or surgery at any time during the process. Also, the medical provider interface 500 can be used to monitor certain parameters of the patient post-surgery. The interface can also be used to cue the patient to certain co-morbidities associated with the patient so they are at the forefront of the surgeon's mind as treatment and follow-up is monitored. The fields and display areas of interface 500 can include data created by the management system 308 performing one or more of methods 700-1300 described herein.

In one embodiment, the medical provider interface 500 may include a display area 504 that lists each patient with a prescribed RFM regimen. Optionally the list may include a scroll bar 516 to provide access to other information. The list may also be sortable, for example, by selecting a sort button 540. The list may include icons that can be selected to provide additional information to the medical provider. In one embodiment, the icons include one or more of a status icon 508, a detail icon 512, and a chat icon 520. Optionally, one or more of the icons 508, 512, 520 may be associated with each patient.

The status icons 508 can graphically indicate the patient's progress in an RFM regimen and readiness for surgery. For example, the status icon 508 may be coded or shaped to indicate different levels of risk or readiness. In one embodiment, the icons are colored or shaped to indicate up to three levels risk or readiness, such as: low 508A, medium 508B, and high 508C. In another embodiment, status icon 508A can indicate the patient has completed an RFM regimen, status icon 508B can indicate that the patient needs attention or that there is a problem with the patient's progress, and status icon 508C may indicate the patient has not completed an RFM regimen. Optionally, status icon 508D can indicate that patient is in post-operative monitoring. The display configuration of the medical provider interface may be altered by a user by selecting an options button 544.

A detail icon 512 can be selected to provide detailed patient information. The chat icon 520 may be used to establish communication with the patient. The medical provider interface 500 may also include scheduling icons 524, 528 to schedule a patient for an office visit and/or for surgery. The medical provider interface 500 can also include links 532, 536 to a primary and secondary condition of the patient. The links 532, 536 may also display the primary and secondary conditions, such as “AFib” and “Hypertension” or other medical conditions of the patient. By selecting links 532, 536, medical literature related to the condition and relevant can be presented to the medical provider.

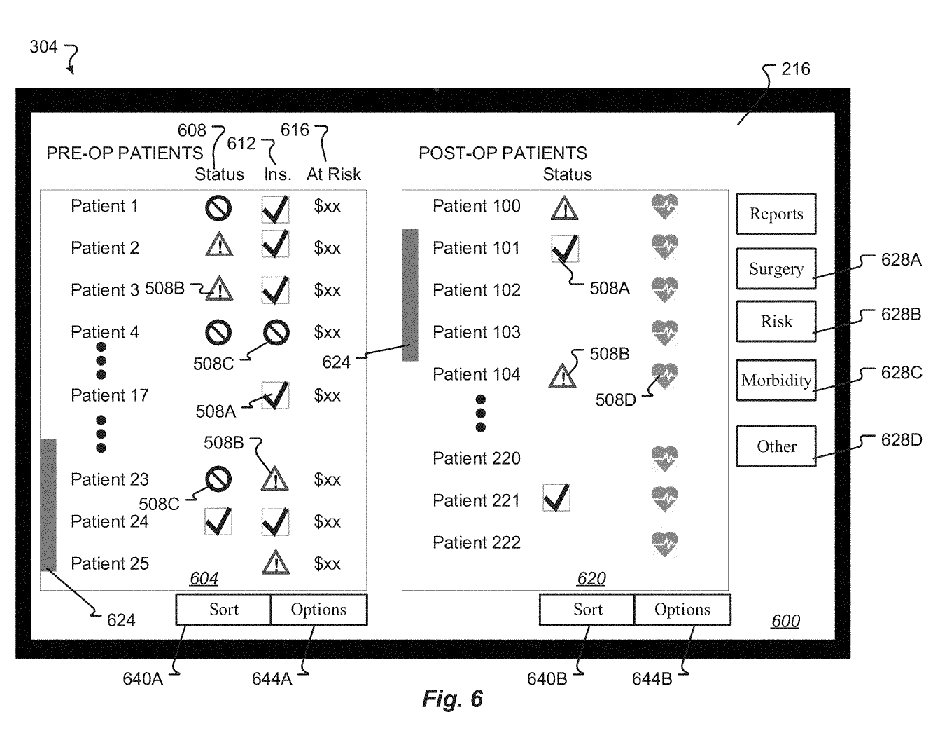

Referring now to FIG. 6, a view of a medical administrator interface 600 is provided. The medical administrator interface 600 may be associated with a user at a hospital, a surgery center, the medical provider (such as a surgeon), or a different entity and may be presented on a display 216 of a service provider system 304. Through this interface, the user can monitor both pre-and post-surgery patients. Information presented by the medical administrator interface 600 can be generated by one or more of methods 700-1300 described herein.

The medical administrator interface 600 can include a first display area 604 and a second display area 620. In one embodiment, the first and second display areas 604, 620 are displayed simultaneously. Alternatively, only one of the first and second display areas 604, 620 is displayed at one time. In this embodiment, the user can select which one of the first and second display areas 604, 620 to display. The first and second display areas can include scroll bars 624 to display more, or different, information. In one embodiment, a sort button 644 can be selected to alter the displays areas 604, 620.

The first display area 604 can present information on pre-operative patients. The first display area 604 enables the user, such as a medical provider, to monitor each patient's progress in an RFM regimen and other information. The patients may be displayed in a scrollable list. The list may be sorted in any manner, such as alphabetically or by progress in a prescribed RFM regimen. Icons 508 may be associated with patients in the first display area 604. The icons 508 may be the same, or similar to, the icons 508 of the medical provider interface 500 described in conjunction with FIG. 5.

Additionally, or alternatively, the icons 508 may be presented in columns related to one or more of: (i) readiness of the patient for surgery; (ii) status of insurance carrier approval of surgery; and (iii) risk amount in a performance-based reimbursement system. For example, the first display area 604 may include a status column 608, an insurance column 612, and an at risk column 616. The status column 608 can include icons 508 to graphically present the patient's progress in a RFM program. Specifically, the icons may indicate the patient is ready (icon 508A), is not ready (508C), or needs attention (508B). Optionally, the icons 508 may indicate the progress of the patient in an RFM regimen as a percentage. In one embodiment, the icons in the status column 608 can be selectable. When an icon 508 in the status column 608 is selected, the service provider system 304 can retrieve information and/or reports related to a patient's progress in an RFM regimen from the records database 324 of the management system 308.

The insurance column 612 can graphically indicate whether an insurance provider or other payor has approved a surgical procedure for a patient, such as with icon 508A. Optionally, the insurance column 612 may include an icons which indicate the surgery is approved (508A), the surgery is not approved (icon 508C), and there is a problem in the approval process (508B). In one embodiment, the icons in the insurance column 612 can be selected to provide more information on the status of approval of surgery by an insurer.

The at risk column 616 may indicate a dollar amount at risk in a performance-based payment system. Thus, the at risk column 616 may indicate an amount of reimbursement an insurance provider will provide for a surgical procedure for the patient. The at risk column 616 may also display a total amount of reimbursement that can be paid for a medical procedure and a second amount that may be paid based on the patient's progress in an RFM regimen. For example, the at risk column 616 can indicate a reduced amount of reimbursement that will be provided for a patient that has not completed a prescribed RFM regimen prior to surgery. Thus, for patient 1 who has not completed an RFM program but who has been approved for surgery, the at risk column 616 can indicate that the insurer will pay a reduced amount for the surgery. In contrast, patient 24 has completed an RFM program and has been approved for surgery. Accordingly, the insurer may reimburse the hospital and medical provider a greater amount for the surgery.

The second display area 620 can present a list of patients in post-operative monitoring. An icon 508A, 508B may be provided to indicate each patient's progress or status in the post-operative monitoring program. Optionally, another icon 508D may be provided that a user may select to receive detained follow-up monitoring data for a patient. The second display area 620 enables a medical provider (or other user) to monitor the patient's status and vital biometrics.

Further, the medical administrator interface 600 facilitates the generation of various comprehensive reports that could be used to assess the provider's outcomes based performance. In this manner, the interface may provide insight into how to improve. The reports can be provided by selecting associated icons. The icons may include, but are not limited to: (i) a first icon 628A to generate a report indicating outcomes by surgery; (ii) a second icon 628B for a report detailing outcomes by risk; and (iii) a third icon 628C for a report related to outcomes by co-morbidity. Other reports are contemplated. For example, by selecting a fourth report icon 628D, a user may select from other predetermined reports. Additionally, the user can generate a custom or user defined report with data from the RFM system by selecting the fourth report icon 628F.

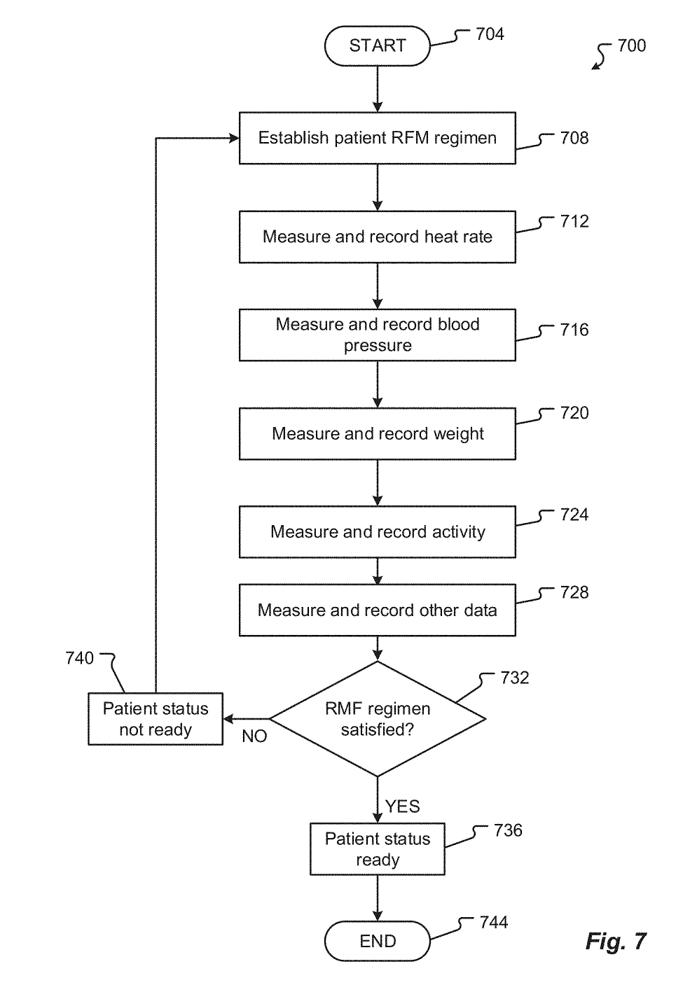

Referring now to FIG. 7, a Pre-Surgery Patient Data Flow is generally illustrated. More specifically, the pre-surgery patient data flow generally illustrates a method 700 for determining if a patient has satisfied a prescribed pre-surgery RFM regimen.

A pre-surgery RFM regimen is established for a patient as described herein in operation 708. In one embodiment, the RFM regimen is created for the patient by a surgeon or other medical provider. When creating the program, the surgeon may create goals, or rules, associated with one or more of the patient's: (i) heart rate; (ii) blood pressure; (iii) weight; and (iv) physical activity. The surgeon can also establish rules for other parameters, such as: body mass index, alcohol use, tobacco use, calorie intake, blood sugar level, AFib burden, and compliance with medical directives (such as use of prescribed medication and/or management of sleep-disordered breathing). The rules may include desired or required values for each element. Additionally, the rules can specify data collection requirements, such as data collection frequency, data collection method (including data required to be entered by the patient), and penalties for failing to comply with the rules. The rules may also give equal weight to each element. Alternatively, one or more elements may have different weights compared to other elements. The rules may be stored in database 316. The surgeon may use a service provider system 304 to enter the rules for the patient RFM regimen into the management system 308.

The method 700 continues to operations 712-728 where patient data associated with each element of the RFM regimen is measured and transmitted to the management system 308. In one embodiment, data such as heart rate, blood pressure, weight, and activity is entered into patient system 332. Other data may be collected and recorded in operation 728. Optionally, at least some of the data is automatically transmitted to patient system 332 by a sensor 336 associated with the patient 328. Additionally, or alternatively, the patient may enter some or all of the data required to be collected for each element into the patient system 332 using an input device 212.

The patient system 332 then sends the data to the management system 308 through the communication network 110. The management system 308 processes the data associated with each patient. Specifically, the data collection module 320 stores the data in a patient record in database 324.

In operation 732, the rules module 312 of the management system 308 can determine, based on the rules stored in database 316 associated with each element, when the patient has satisfied the prescribed RFM regimen. For example, in one embodiment, when the patient's heart rate, blood pressure, weight, and activity comply with levels established by the patient's surgeon, management system 308 may determine that patient has completed the prescribed RFM regimen. Regardless, when management system 308 determines the patient has completed the prescribed regimen, the method proceeds “YES” to operation 736 to update the patient's surgery status to “Ready” in database 324. Method 700 then proceeds to END operation 744. Optionally, method 700 may continue to one or more of methods 800-1300 described herein.

Alternatively, if the management system 308 and/or the rules module 312 determines the patient has not completed the prescribed RFM regimen, the method may return “NO” to operation 740. In operation 740, the management system 308 may update or maintain the patient's status to “Not Ready” in database 324. Method 700 may then return to operation 708 and continue to measure and transmit data to the management system 308. In one embodiment, a status flag, data field, or icon associated with each patient is updated by management system 308 each time the data collection module 320 receives data. In this manner, the status flag in a patient's record indicates whether the patient is “Ready” or “Not Ready” for surgery.

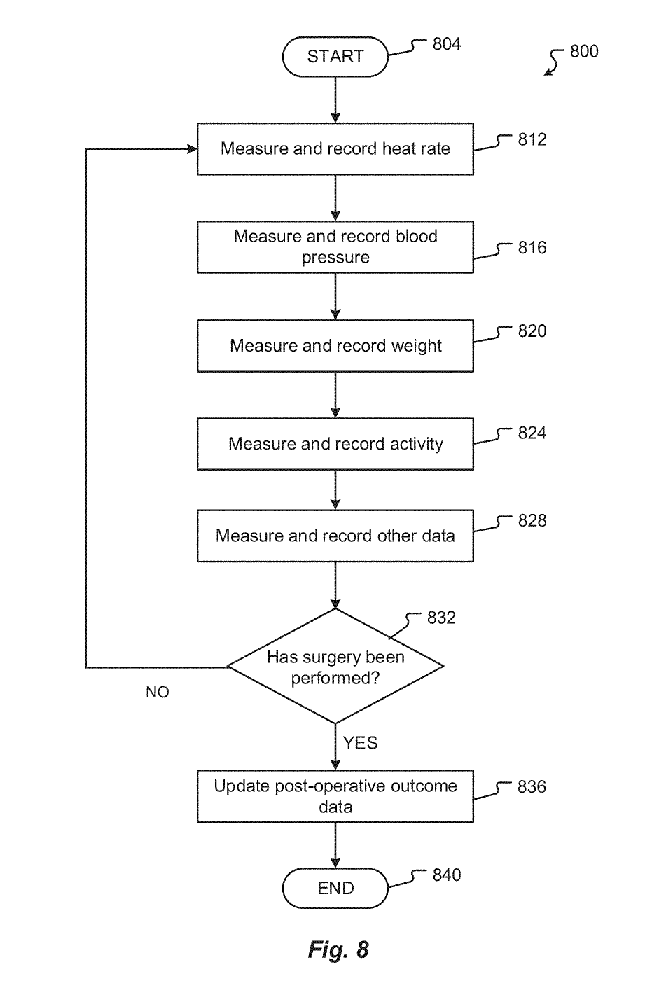

Referring now to FIG. 8, a Post Surgery Patient Data Flow method 800 of the present invention is generally illustrated. Data similar to pre-surgery data described in conjunction with method 700 of FIG. 7 is collected by the patient system 332 in operations 812-828. More specifically, method 800 of FIG. 8 continues measuring and transmitting data associated with each element of the RFM regimen to the management system 308 in a manner the same as, or similar to, operations 712-728 of method 700.

In one embodiment, data associated with one or more elements of the patient's RFM regimen is collected by the patient 328. Additionally, or alternatively, some or all of the data may be automatically collected by sensors 336 of associated with the patient system 332. The patient system 332 then transmits the data through the communication network 110 to the management system 308.

In operation 832, if a patient status flag is set to “Not Ready” in a record of database 324, method 800 returns “NO” and the method returns to operation 812 to receive patient data and transmit the patient data to the management system 308. Alternatively, when the status flag is set to “Surgery Complete,” then method 800 continues “YES” to operation 836.

At operation 836, the management system 308 prepares (or updates) a post-operation report. The post-operative report may include patient data and outcomes data which is collected and associated with the patient's records by the data collection module 320 and stored in database 324. The management system 308 may also send the post-operation report to the rules module 312. The rules module 312 can use results of the surgery to review the rules stored in database 316. In this manner, the rules module 312 may revise, add, or remove one or more rules stored in database 316 to account for a surgical outcome.

Method 800 may then continue to END operation 840. Optionally, method 800 may continue to one or more of methods 700 and 900-1300 described herein.

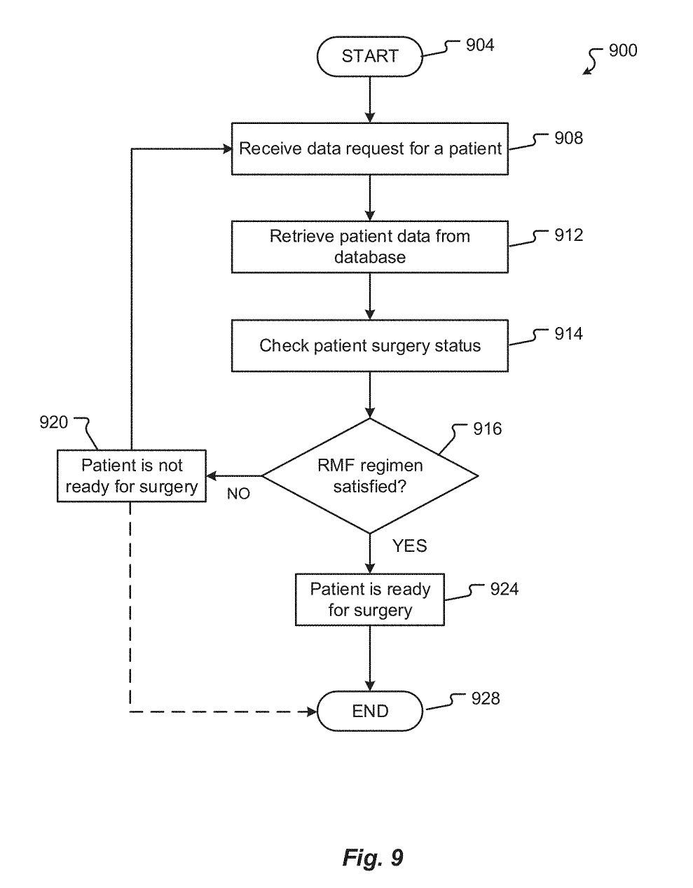

Referring now to FIG. 9, a method 900 of determining whether a patient is ready for surgery is generally illustrated. In operation 908, the RFM system 300 can receive an input, such as a request for information from a user. The user may be a medical provider. The input may comprise a request for the status of a patient or a request for a report. The input may be entered with an input device 212 of a service provider system 304. For example, in one embodiment, the input may comprise a selection of a detail icon 512 associated with a patient that is displayed in a medical provider interface 500 described in conjunction with FIG. 5. In another embodiment, the input comprises a request for information for a plurality of patients. For example, when a medical provider opens a medical provider interface 500, the service provider system 304 may transmit a request to the management system for information on all patients of the medical provider. In this manner, the management system 308 can provide information to the service provider system 304 required to update the icons 508 displayed in the medical provider interface 500.

Optionally, in another embodiment, the input may be generated by a patient 328 using a patient system 332. The patient 328 may open a patient user-interface 400 on the patient system 332. In response, the management system 308 can review the patient's medical record to update one or more fields of the patient user-interface 400. For example, in management system 308 can determine the patient's percent readiness for surgery to generate the progress display area 424 which is generally illustrated in FIG. 4.

The input is transmitted by the communication network 110 to the management system 308. In operation 912, in response to receiving the input, the management system 308 can retrieve data, such as a record, related to the patient from database 324. Optionally, a data collection module 320 can retrieve the record for the management system 308.

In operation 914, the management system 308 can check the patient's status. In one embodiment, the management system 308 may poll the “surgery status flag” or field of a record associated with the patient.

The management system 308 then determines if the patient is ready for surgery, such as by completing an RFM regimen, in operation 916. In one embodiment, if the surgery status field of a record associated with the patient includes “ready,” the management system 308 returns “True.” Alternatively, the management system 308 returns “false” if the patient is not ready. Other non-binary parameters may be returned by the management system 308, such as a percentage readiness encapsulated in a floating-point variable ranging from 0.00 to 1.00. In one embodiment, the rules module 312 can determine the patient's progress in an RFM regimen according to method 700. The rules module 312 can consider data collected by sensors 336 and recorded in the patient record stored in database 324. The rules module 312 can compare the stored sensor data to rules stored in database 316 to determine if the patient has completed the RFM regimen. In one embodiment, the rules module 312 can determine a percent complete for the patient.

If the patient is not ready for surgery, method 900 proceeds NO to operation 920. The management system 308 can provide the patient's status for surgery and, optionally, the patient's progress in the RFM regimen to the service provider system 304 or the patient system 332 from which the request for status originated. The patient's progress may be expressed as a percentage. Method 900 can then return to operation 908 to wait for another request. Optionally, method 900 may continue to END operation 928.

When the patient is ready for surgery, method 900 proceeds YES to operation 924. The management system 308 can provide the patient's status for surgery and completion of the RFM regimen to the service provider system 304 or the patient system 332 which generated the request in operation 908. Method 900 then continues to END operation 928. Optionally, the method may proceed to one or more of methods 700, 800, and 1000-1300.

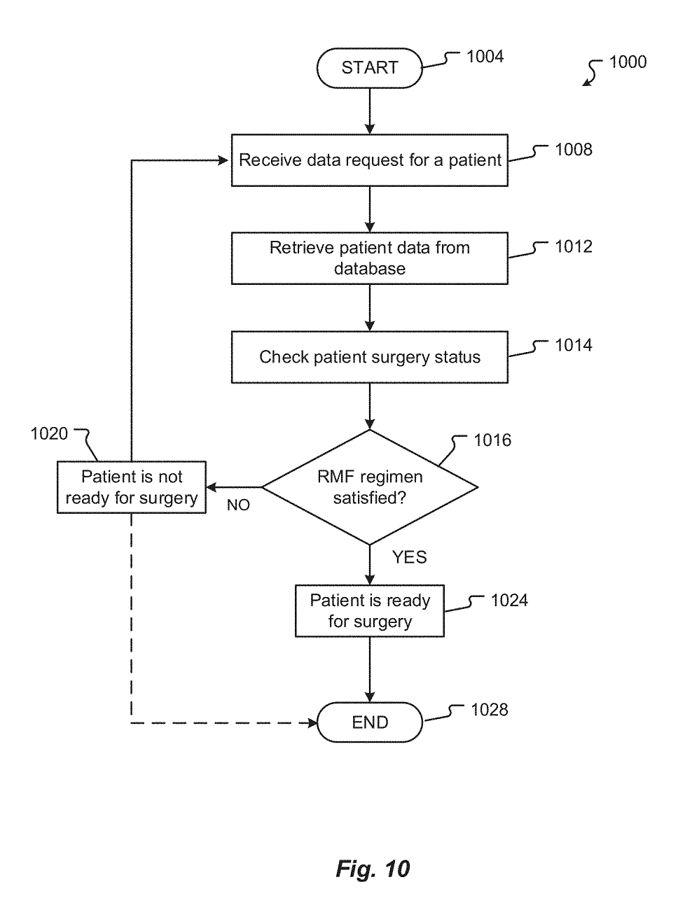

FIG. 10 illustrates a method 1000 similar to the method 900 of FIG. 9. Accordingly, the operations of method 1000 are the same as, or similar to, the operations of method 900. Notably, in method 1000, an insurance provider may request the status of a patient from the RFM system 300. The request may be entered into a service provider system 304. The request can originate when an insurance provider opens an administrator interface 600 or a similar interface. The service provider system 304 may then generate a request for information for all patients required to generate the administrator interface 600.

In one embodiment, the insurance provider interacts with the RFM system 300 from a service provider system 304 suited for his or her needs as an insurance provider. For example, in one embodiment, a service provider system 304 for an insurance provider has different functionality compared to a service provider system 304 for a doctor or a medical administrator. All elements of the RFM system 300, including each patient system 332, service provider system 304, and management system 308, administer appropriate authentication to ensure HIPPA compliance.

In response to receiving an input from the insurance provider in operation 1008, the management system 308 may retrieve patient data in operation 1012 and check the patient's surgery status in operation 1014.

In operation 1016, the management system can determine if the patient is ready for surgery. Notably, the management system 308 may use different or additional criteria for determining if the patient is ready for surgery compared to method 900 of FIG. 9. For example, the insurance provider may require a higher level of compliance with the elements of the patient's RFM regimen. Additionally, or alternatively, the insurance provider may request that the patient comply with one or more elements of the RFM regimen. Further, method 1000 of FIG. 10 may assign different weights to one or more elements of the RFM regimen compared to the method 900 of FIG. 9.

The insurance provider may also adjust a deductible that the patient is required to pay for a surgical procedure based at least in part on the patient's progress in the RFM regimen. For example, the insurance provider may reduce the patient's deductible based on positive progress of the patient related to elements of the RFM regimen. Additionally, or alternatively, the insurance provider may increase the patient's deductible in response to non-compliance with one or more elements of the RFM regimen.

In another example, the insurance provider may alter an amount of reimbursement that will be paid to a medical provider or hospital for a surgical procedure based at least in part on progress of the patient in the RFM regimen. In one example, the insurance provider may reduce the amount of reimbursement to one or more of the medical provider and a medical facility when the patient has not successfully completed a prescribed RFM regimen prior to surgery. Alternatively, the insurance provider may increase reimbursement to one or more of the medical provider and the medical facility if surgery is performed after the patient has successfully complied with all elements (or selected elements) of the RFM regimen before surgery is performed.

In operation 1016, the method 1000 may continue “YES” to operation 1024 after the management system 308 determines the patient's status is “Ready” for surgery. The management system can provide the patient's status to the insurance provider's system 304 in operation 1024. Alternatively, if the management system 308 determines the patient is “Not Ready” for surgery, method 1000 can return “NO” to operation 1020. In operation 1020, the management system provides the patient's status to the insurance provider's system 304. The patient's status may be reported as a percentage of completion of the RFM regimen. In one embodiment, method 1000 returns to operation 1008 and waits for another insurance provider input. Alternatively, method 1000 may optionally continue to END operation 1028. In one embodiment, method 1000 can continue with one or more of methods 700-900 and 1100-1300.

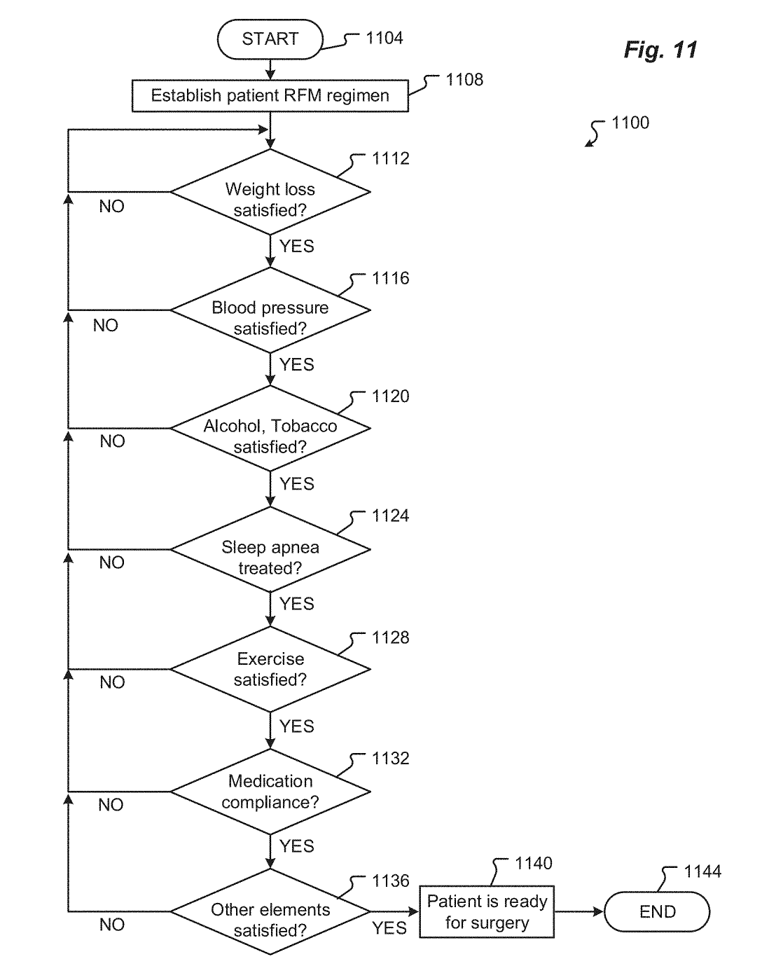

Referring now to FIG. 11, an embodiment of another method 1100 for determining if a patient is ready for surgery is generally illustrated. Method 1100 is similar to method 700; however, method 1100 provides a more detailed flow of how a patient would move through a process to achieve a ready status for an atrial fibrillation catheter ablation procedure administered by a Cardiac Electrophysiologist. Method 1100 generally illustrates elements of a RFM regimen for a patient that may be prescribed by a medical provider for the patient.

In operation 1108, a RFM regimen is established for the patient. The RFM regimen can be established in the same, or similar, manner as described in operation 708 of method 700. Elements of the RFM regimen may be determined by rules stored in database 316. In one embodiment, one or more of the rules 316 may be selected from parameters described in an article in the Journal of American College of Cardiology by Doctor Prashanthan Sanders et al., entitled “Aggressive Risk Factor Reduction Study for Atrial Fibrillation and Implications for the Outcome of Ablation, The ARREST-AF Cohort Study”, which is incorporated herein by reference in its entirety.

When selecting elements of the RFM regimen for the patient, the medical provider may also establish rules that describe attributes of the patient that must be modified, and to what levels, prior to performing catheter ablation on the patient to achieve the best clinical outcomes. The elements of the RFM regimen may include, but are not limited to, one or more of: (i) weight loss; (ii) a certain maximum blood pressure; (iii) a specified alcohol or tobacco use limitation; (iv) treatment of sleep apnea; (v) a required amount of exercise; and (vi) medication compliance. The RFM regimen can optionally include other elements.

The management system 308 stores the patient's RFM regimen in database 324. In operation, the management system 308 may receive patient data related to the elements of the RFM regimen. More specifically, sensors 336 of a patient system 332 can collect data relevant to the RFM regimen. The data may be collected by the sensors 336 at times specified by the rules stored in database 316. The patient system 332 can transmit the data to the management system 308. The data collection module 320 of the management system can then update the patient's record in the database.

The management system 308 can then determine whether or not the patient has completed the RFM regimen using rules associated with each element. More specifically, the rules module 312 can review a patient's record stored in database 324 to determine if the patient has completed one or more requirements of an RFM regimen in operations 1112-1136. For each operations 1112-1136 the sensors 336 can collected and send data to the management system 308. The data collections module 320 can update the patient's record in database 324 with the data.

In one embodiment, when the management system 308 determines the patient has not completed one or more elements of the RFM regimen in operations 1112-1136, method 1100 returns “NO” to operation 1112. For example, in operation 1112, the rules module 312 determines if the patient has lost a prescribed amount of weight. In operation 1116, the management system determines if the patient has achieved a prescribed blood pressure. Alcohol and tobacco use are checked for compliance with the RFM regimen in operation 1120. Treatment of the patient's sleep apnea, if necessary, is determined in operation 1124. Exercise requirements and medication compliance are determined in operations 1128 and 1132. Other requirements of the RFM regimen can be evaluated in operation 1136.

For each element 1112-1136 of the patient's RFM regimen that is not complete, method 1100 returns until more data related to the patient is received from the sensors 336 of the patient system 332. Additionally, or alternatively, the management system 308 may update a record associated with the patient indicating the patient's status is “NOT READY” as previously described, for example, in conjunction with method 700. In one embodiment, the management system 308 may determine a percent complete for the patient. More specifically, the percent complete may be from 0.00 to 1.00 and determined based on the data collected for each element of the patient's prescribed RFM regimen. Method 1100 repeatedly assesses a patient on all risk factor criteria of the RFM regimen according to the rules stored in database 316 until all criteria of the RFM regimen have been met or completed. In one embodiment, the rules in database 316 are based on the ARREST-AF article and the LEGACY article published in the Journal of the American College of Cardiology which are described above and incorporated herein.

When all requirements of the RFM regimen are completed, method 1100 proceeds YES to operation 1140. In operation 1140, the management system 308 updates the patient's status in database 324 to “Ready for Surgery”. Optionally, the management system 308 can send the updated status to one or more of a service provider system 304 and a patient system 332. In this manner, the patient's status in one or more of user-interfaces 400, 500, 600 can be updated for a user, such as the patient, a doctor, a medical administrator, and an insurance provider. Method 1100 can then proceed to END operation 1144. Optionally, the method may continue into one or more of methods 700-1000 and 1200-1300.

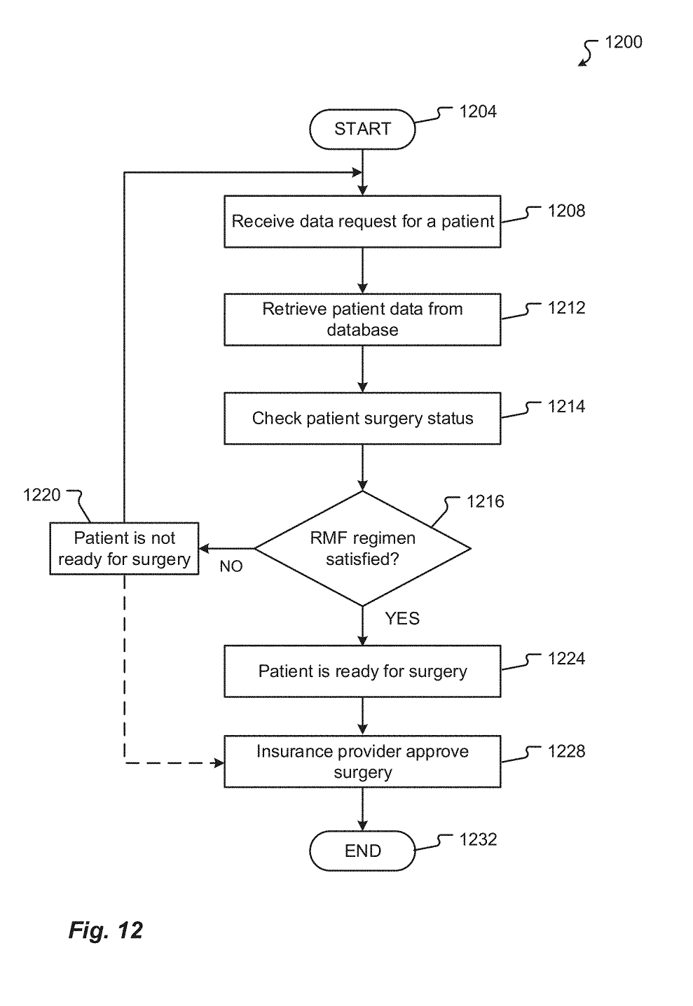

FIG. 12 illustrates still another flow or process diagram of a method 1200 for approval of surgery by an insurance provider using the RFM system 300 of FIG. 3. Operations 1208-1224 are the same as, or similar to operations X08-X24 of methods 900 and 1000. Notably, method 1200 facilitates surgery approval by an insurance provider. The insurance provider may use a service provider system 304 in communication with management system 308 to receive data from the RFM system 300. Specifically, method 1200 illustrates one embodiment of how an insurance provider may use patient data retrieved from database 324 and/or rules stored in database 316 to approve the patient for surgery.

In operation 1208, the insurance provider provides an input with a service provider system 304 to determine the status of a patient's readiness for surgery, to have the best chance at a successful procedure. The input may be a selection of an icon 508 in a user interface displayed by the service provider system 304, such as a user interface 600 and the like. The management system 308 retrieves relevant patient data from database 324 in operations 1212 and checks the patient's readiness for surgery in operation 1214.

At operation 1216, the management system 308 can determine whether the patient has completed a prescribed RFM regimen. The rules module 312 can evaluate patient data from database 324 using the rules in database 316. The management system 308 can then indicate whether the patient is ready or not ready for surgery. In one embodiment, a patient may be approved for surgery in operation 1216 although one or more elements of an RFM regimen are incomplete or only partially complete. Accordingly, the value returned by the management system may not result in a binary outcome of approved or not approved for surgery. More specifically, in one embodiment, the management system 308 may return a floating point variable ranging from 0.00 to 1.00 based on the patient's progress in a RFM regimen. This floating point variable may be used by the insurance provider to calculate deductibles appropriately for the patient.

If the patient is not ready for surgery, method 1200 proceeds NO to operation 1220. Alternatively, if the patient is ready for surgery, method 1200 continues YES to operation 1224. In operation 1220, the management system 308 can then provide the patient's status to the service provider system 304. Method 1200 can then return to operation 1208 and wait for another request for data. Optionally, method 12 can continue from operation 1220 to operation 1228.

At operation 1224, the management system 308 can update the patient status to ready for surgery. Method 1200 then proceeds to operation 1228. The insurance provider can then review the patient's record and, optionally, approve surgery or another medical procedure for the patient. Optionally, the insurance provider can adjust the amount of a deductible paid by the patient for the procedure. For example, if the patient has completed an RFM regimen, the insurance provider can reduce the amount of the patient's deductible. Alternatively, if surgery has been approved for a patient who has not completed an RFM regimen, the insurance provider can increase the amount of the patient's deductible. In one embodiment, for a patient who has not completed an RFM regimen, the patient's deducible may be increased by dividing the deductible by a percentage representing the patient's progress in the RFM regimen. Accordingly, if the patient's deductible is $500, but the patient is 60% ready for surgery according to an established RFM regimen and the return value of 0.60 from the management system 308, then the new deductible may be computed as $500 divided by 0.60 resulting in a $833.33 new deductible for the patient. This is just one example of how the readiness for surgery return value from management system 308 could be used by an insurance provider.

Similarly, in another example, the patient's progress in the RFM regimen may be used to determine a reduction in an amount of reimbursement paid by an insurance provider to a medical facility or medical provider for a medical procedure performed before the RFM regimen is completed by the patient. In one embodiment, the insurance provider may only reimburse the medical facility or medical provider an amount in proportion to a percentage equal to the patient's progress in the RFM regimen. In this manner, if a patient is only 60% complete with the RFM regimen when a medical procedure is performed, the insurance provider may only reimburse 60% of the cost of the surgery of the medical procedure.

Method 1200 then proceeds to END operation 1232. Optionally, the method may continue with one or more of methods 700-1100 and 1300.

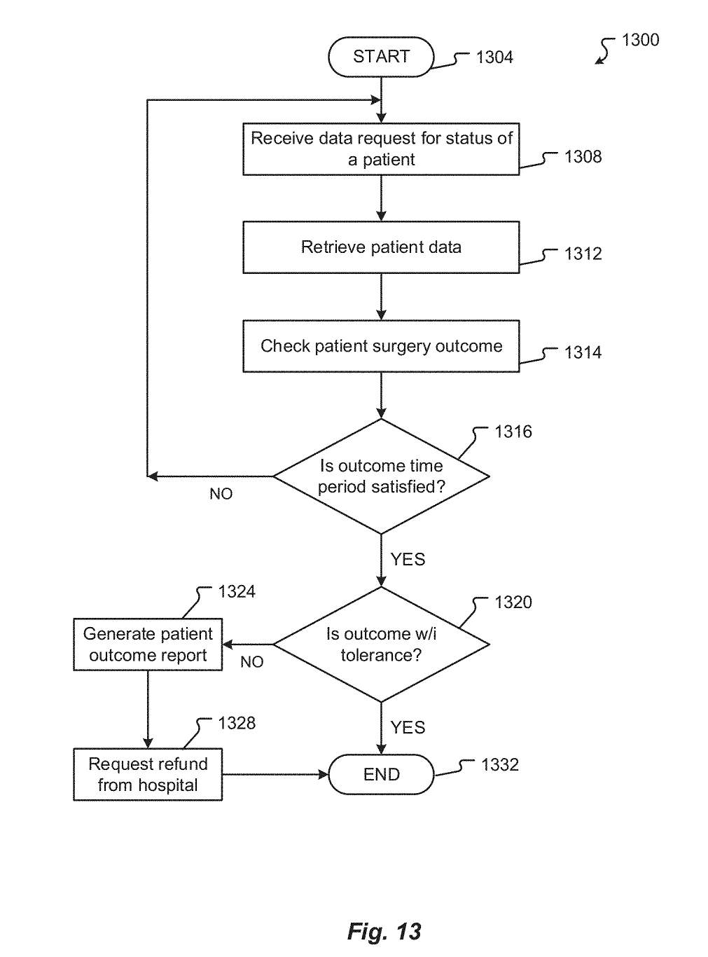

Referring now to FIG. 13, still another method 1300 of the present invention related to determining whether an outcome of surgery performed on a patient meets established expectations of care is generally illustrated. Method 1300 describes a scenario for which performance based payment from insurance providers to medical providers is widespread. In method 1300, the insurance provider can periodically or up to a certain point in time, check the outcome status of a patient using a service provider system 304 in communication with the RFM system 300.

In operation 1308, an insurance provider can request information on a surgical outcome for a patient. The request can be entered in a service provider system 304. The service provider system transmits the request to the management system 308 through network 110.

In response to input from the insurance provider, the RFM system 300 can query the management system 308. More specifically, in operation 1312, the management system 308 can retrieve patient data from database 324. In operation 1314, the management system 308 can review the results of the surgery. Additionally, the management system 308 can review data collected on the patient after the surgery using data stored in the patient's record in the database 324. In one embodiment, patient data from sensors 336 which is collected after the surgery is stored in database 324.

In operation 1316, the management system 308 can determined if an outcome time period is satisfied. For example, there may be a predetermined period of time after a surgery within which the management system will evaluate the patient. If the time period is not satisfied (or has not expired), method 1300 returns NO to operation 1308. Alternatively, if the time period is satisfied (or has expired), method 1300 continues YES to operation 1320.

In operation 1320, the management system 308 can determine whether the outcome of a surgical procedure performed on a patient is within an expected tolerance. For example, the management system 308 can consider patient data in a patient record to determine if the patient's condition after surgery is within the expected tolerance. In one embodiment, the rules module 312 may use rules stored in database 316 to determine if the patient's outcome is within the expected tolerance. Additionally, or alternatively, the outcome of the surgery can be compared to a tolerance based on published medical literature. If the outcome of the surgery is within the expected tolerance, method 1300 continues YES to end operation 1332. Alternatively, method 1300 proceeds NO to operation 1324.

In operation 1324, the management system 308 can generate a report related to the surgical outcome of the patient. The report may be used to update a field or icon in one or more of user interfaces 400, 500, 600 and the like.

Based on the return value from the management system 308, the insurance provider may request a refund from at least one of a medical provider who performed the surgery and a hospital in operation 1328. Specifically, the insurance provider can us data in the report generated in operation 1324 to alter reimbursement paid to the medical provider or the hospital in accordance with a performance based payment contract. In this manner, the insurance provider can obtain rebates (or refunds) for procedures that were not within certain successful outcome tolerances. This paradigm of accountability offers many benefits. For example, by facilitating the use of a performance based payment contract, the RFM system of the present invention may help reduce healthcare costs in the United States by linking insurance reimbursement to surgical results, such as outcome improvement for the patient.

All methods described herein in conjunction with FIGS. 7-13 may include more or fewer steps or can arrange the order of the steps differently than those shown. Additionally, although the steps are generally illustrated sequentially, they may be performed substantially simultaneously. The methods can be executed as a set of computer-executable instructions executed by a computer system or processor and encoded or stored on a computer readable medium. In other configurations, the methods may be executed by a series of components, circuits, gates, etc. created in a hardware device, such as a System of Chip (SOC), Application Specific Integrated Circuit (ASIC), and/or a Field Programmable Gate Array (FPGA). Also, while the flowcharts of FIG. 7-13 have been discussed and illustrated in relation to a particular sequence of events, it should be appreciated that changes, additions, and omissions to this sequence can occur without materially affecting the operation of the disclosed embodiments, configuration, and aspects. Further, any of the steps, functions, and operations discussed herein can be performed continuously and automatically.

Optionally, the RFM system and method of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The RFM system described herein can relate to communications systems and/or devices and may be capable of communicating with other devices and/or to an individual or group of individuals. Further, the RFM system can receive user input in unique ways. The overall design and functionality of the RFM system provides for enhanced surgical outcomes for patients and improved patient management by medical providers. As described herein, the RFM system may be electrical, mechanical, electro-mechanical, software-based, and/or combinations thereof.

The exemplary systems and methods of this disclosure have been described in relation to a RFM system and associated devices. However, to avoid unnecessarily obscuring the present disclosure, the preceding description omits a number of known structures and devices. This omission is not to be construed as a limitation of the scopes of the claims. Specific details are set forth to provide an understanding of the present disclosure. It should however be appreciated that the present disclosure may be practiced in a variety of ways beyond the specific detail set forth herein.

Furthermore, while the exemplary aspects, embodiments, options, and/or configurations illustrated herein may show various components of the RFM system 300 collocated, certain components of the RFM system can be located remotely, at distant portions of a distributed network, such as a LAN and/or the Internet, or within a dedicated system. Thus, it should be appreciated, that the components of the RFM system can be combined in to one or more devices, such as a Personal Computer (PC), laptop, netbook, smart phone, Personal Digital Assistant (PDA), tablet, etc., or collocated on a particular node of a distributed network, such as an analog and/or digital telecommunications network, a packet-switch network, or a circuit-switched network. It will be appreciated from the preceding description, and for reasons of computational efficiency, that the components of the RFM system 300 can be arranged at any location within a distributed network of components without affecting the operation of the RFM system. For example, the various components can be located in a switch such as a PBX, gateway, in one or more communications devices, at one or more users' premises, or some combination thereof. Similarly, one or more functional portions of the RFM system 300 could be distributed between a telecommunications device(s) and an associated computing device.

Furthermore, it should be appreciated that the various links connecting the elements of the RFM system 300 can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

A number of variations and modifications of the disclosure can be used. It would be possible to provide some features of the disclosure without providing others. The RFM system 300 and method of the present disclosure could be added to an existing data system or medical information system.

The present disclosure, in various aspects, embodiments, and/or configurations, includes components, methods, processes, systems and/or apparatus substantially as depicted and described herein, including various aspects, embodiments, configurations embodiments, sub combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Examples of the processors that may be used in the RFM system 300, as described herein, may include, but are not limited to: at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3560K 22 nm Ivy Bridge, the AMD® FX™ family of processors, AMD® FX-4300, FX-6300, and FX-8350 32 nm Vishera, AMD® Kaveri processors, ARM® Cortex-A and ARM926EJ-STM processors, other industry-equivalent processors, and may perform computational functions using any known or future-developed standard, instruction set, libraries, and/or architecture.

The above aspects of the present disclosure have a variety of advantages. First, patients may be motivated to participate in a RFM regimen prescribed by a medical provider. Completing the program may improve the patient's health and improve the result of surgery performed on the patient. Further, insurance providers may reduce, or eliminate, deductibles or co-payment amounts for patients who successfully complete a prescribed RFM regimen. Alternatively, insurance providers may increase deductibles and co-payments for patients who have not completed, or have only partially completed, a RFM regimen. In one embodiment, an insurance provider may decrease reimbursements to medical providers and hospitals for surgery performed on a patient who has not be prescribed a RFM regimen, or has not completed a prescribed RFM regimen. Similarly, an insurance provider may decrease reimbursement, or obtain a refund, from a hospital or medical provider when the post-operative outcome of a patient is not within an expected outcome or tolerance. Medical providers may achieve better surgical results for their patients. Insurance cost for medical providers may decrease. Also, medical providers may improve collections in a performance based payment regime. Insurance providers may benefit when insured patients have better surgical outcomes with reduced post-operative complications or the need for additional medical procedures.

What is claimed is:

1. A system for monitoring a plurality of health parameters associated with a patient to improve a surgical outcome, comprising:
a data monitor for the patient, including at least one of the following:
a patient blood pressure data measurement device;
a patient weight data measurement device;
a patient physical activity data monitoring device;
a patient heart rate data monitor;
a patient blood alcohol data measurement device; and
a patient nicotine presence data measurement device;
a control unit to receive data from the patient, wherein the patient requires a surgical procedure;
wherein the control unit is configured to store a pre-surgery regimen prescribed for the patient; and
wherein the control unit is further configured to provide a recommendation as to the patient's readiness for the surgical procedure.

2. The system of claim 1, wherein the data monitor is an implantable medical device.

3. The system of claim 1, wherein the control unit is further configured to receive data related to the patient after the surgical procedure has been performed.

4. The system of claim 3, wherein the control unit generates a post-surgery patient status report.

5. The system of claim 3, wherein the control unit determines if the results of the surgery were acceptable.

6. The system of claim 1, wherein the pre-surgery regimen includes at least one of:
a dietary regimen, an exercise program, a medication protocol, a target blood pressure, a target blood glucose level, lipid management, arrhythmia management, a reduction in alcohol consumption, a reduction in tobacco use, a target weight, a target level for at least one hormone, and a target body mass index.

7. The system of claim 1, wherein the control unit is a mobile device such as a smart-phone or a tablet computer.

8. The system of claim 1, wherein the control unit is a computer server accessible remotely through the interne.

9. The system of claim 1, wherein the pre-surgery regimen is prescribed by at least one of a medical provider and an insurance provider.

10. The system of claim 1, wherein the pre-surgery regimen is generated by the control unit.

11. The system of claim 1, wherein the control unit is configured to provide at least one of an exercise program, a medication regimen, and a meal plan for the patient.

12. The system of claim 1, wherein the control unit is configured to generate at least one user interface for a user.

13. The system of claim 12, wherein the user interface comprises at least one of:
a patient user interface configured to display the patient's progress in the pre-surgery regimen; and
a surgeon interface configured to display information associated with a plurality of patients and a progress of each of the plurality of patient in a pre-surgery regimen.

14. The system of claim 1, wherein the control unit compares patient data to information stored in a database and provides at least one of: a recommended blood pressure, a recommended weight, and a recommended body mass index for the patient to achieve prior to the surgical procedure.

15. A method of improving the success of a surgical procedure for a patient, comprising:
providing a health regimen for a patient who requires surgery, the health regimen designed to improve the patient's health and reduce risk associated with surgery;
storing, by a control unit, the health regimen provided for the patient;
receiving data from a data monitor related to the patient, the data monitor operable to measure at least one of: (i) blood pressure; (ii) weight; (iii) physical activity; (iv) heart rate data; (v) blood alcohol data; (vi) nicotine presence; (vii) glucose levels; (viii) hormone levels; and (ix) cholesterol levels; and
providing a summary as to the patient's readiness for the surgery.

16. The method of claim 15, further comprising generating a report after the surgical procedure is performed on the patient.

17. The method of claim 15, further comprising providing at least one of a dietary regimen, an exercise program, and a medication regimen to the patient.

18. A non-transitory computer readable media having stored thereon instructions, which when executed by a processor of a management system, cause the processor to perform a method of monitoring a plurality of health parameters associated with a patient to improve a surgical outcome, comprising:
an instruction to store, by the management system, a health regimen for the patient, the health regimen designed to improve the patient's health and reduce risk associated with surgery;
an instruction to receive data from a data monitor related to the patient, the data monitor operable to measure at least one of: (i) blood pressure; (ii) weight; (iii) physical activity; (iv) heart rate data; (v) blood alcohol data; and (vi) nicotine presence; and
an instruction to determine when the patient is ready for surgery.

19. The non-transitory computer readable media of claim 18, further including an instruction for the management system to review patient data and provide one or more of a dietary regimen, an exercise program, and a medication protocol for the patient.

20. The non-transitory computer readable media of claim 18, further including an instruction to generate a user interface for one or more of the patient and a medical provider, the user interface including information on the patient's progress in the pre-surgery regimen.
